(19) United States
(12) Patent Application Publication
(10) Pub. No.: US 2016/0040142 A1
(43) Pub. Date: Feb. 11, 2016

(54) CSF1 THERAPEUTICS

(71) Applicant: UNIVERSITY COURT OF THE UNIVERSITY OF EDINBURGH, Lothian (GB)

(72) Inventors: Stuart Forbes, Lothian (GB); David Hume, Lothian (GB); Ben Stutchfield, Lothian (GB); Deborah Gow, Lothian (GB); Graeme Bainbridge, Kalamazoo, MI (US); Theodore Oliphant, Kalamazoo, MI (US); Thomas L. Wilson, Kalamazoo, MI (US)

(21) Appl. No.: 14/770,767

(22) PCT Filed: Feb. 28, 2014
(86) PCT No.: PCT/GB2014/050595

§ 371 Date: Aug. 26, 2015

(30) Foreign Application Priority Data
Feb. 28, 2013 (GB) 1303537.3
Nov. 27, 2013 (GB) 1320894.7

(51) Int. Cl. C12N 9/12

(52) U.S. Cl. CPC C12N 9/12; C12Y 207/10001; C07K 2319/30; A61K 38/00

(57) ABSTRACT

The present invention provides of compositions of matter comprising a CSF-1 fusion protein and methods of using the same in enhancing regeneration or restoring function of an injured liver. The compositions of matter are useful in the treatment of hepatic disorders, for example, in the prevention and/or treatment of liver disease and to improve outcomes following liver surgery.

The present invention relates to compositions of matter and methods of using the same in enhancing regeneration or restoring function of an injured liver. The compositions of matter are useful in the treatment of hepatic disorders, for example, in the prevention and/or treatment of acute or chronic liver disease or as a supportive therapy to improve the outcomes following liver resection or liver transplantation.

### BACKGROUND

Liver disease is a major cause of morbidity and mortality worldwide but despite this there is currently no effective therapy to enhance regeneration of the diseased or injured liver. A therapy to enhance regeneration of the liver could be applied across a range of medical and surgical contexts for indications including acute, acute-on-chronic or chronic liver failure. In the medical setting acute liver failure can arise from a range of aetiologies, but most commonly due to infection (viral hepatitis), alcohol ingestion, or toxin overdose (such as Paracetamol® overdose). In acute liver failure widespread necrosis of the liver tissue may occur which can rapidly result in death. Acute liver failure can arise on a background of chronic liver disease (acute-on-chronic) where pre-existing liver disease (due to viral hepatitis, alcohol, non-alcoholic fatty liver disease and other causes) further impairs the liver's ability to regenerate. Chronic liver failure can result from a gradual deterioration in liver function (causes as above) until the point at which the liver is unable to maintain homeostasis. In life threatening liver failure the only option is liver transplantation, however the shortfall between potential donors and recipients means many patients will die while awaiting liver transplantation.

Liver regeneration is a complex process involving many growth factors, cytokines and cell types. Liver macrophages perform a range of vital homeostatic roles and are critical to effective liver regeneration. Macrophage colony stimulating factor (M-CSF) also referred to as colony stimulating factor 1 (CSF1) and used interchangeably, is expressed in the liver and is the principle factor responsible for production and maintenance of cells of the monocyte/macrophage lineage, including liver macrophages. Depletion of macrophages and deficiency of CSF1 lead to impaired liver regeneration following partial hepatectomy. It is known from the prior art in a M-CSF null mouse model after partial hepatectomy that M-CSF induced Kupffer cells play a key role in liver regeneration (Amemiya et al., J.Surg. Res. 165, 59-67, 2011). However the potential of CSF1 supplementation to enhance liver regeneration has hitherto not been considered.

A therapy to enhance regeneration and/or restore function of the liver could be applied across a range of medical and surgical contexts for indications including acute, acute-on-chronic or chronic liver failure and would offer immediate benefit to patients, clinicians and health services alike.

A therapy to enhance regeneration of the liver could be applied as a rescue therapy to facilitate regeneration following transplantation or in the context of overwhelming failure or used to prevent decline in chronic liver disease would offer immediate benefit to patients, clinicians and health services alike.

### BRIEF SUMMARY OF THE DISCLOSURE

According to a first aspect of the invention there is provided a biologically active fragment of CSF1 protein or a homolog or a variant or derivative thereof for use in enhancing liver regeneration and/or restoring liver function and/or modulating liver homeostasis.

Also include is the nucleic acid encoding the biologically active fragment of CSF1 protein or a homolog or a variant or derivative thereof.

The inventors have surprisingly found that administration of additional or extra or supplemental CSF-1 to subjects having normal CSF-1 levels increases the size of the liver in healthy animals and improves the ability to repair the liver following loss of function from various causes. It was an unexpected finding that a supplement of CSF-1, to already functioning CSF-1 in an individual, would improve hepatic regeneration or function. The liver is under very strict homeostasis and to date no agent has been identified that can successfully modulate hepatic homeostasis in the clinical setting and increase the size of liver above the normal relative total body weight. However, the present invention provides evidence for use of CSF-1 as an appropriate hepatic trophic and homeostatic agent in mammalian species. Furthermore, the present invention is based upon the observation that CSF-1 can restore the phagocytic capacity of the liver, and thus use of CSF-1 proteins for restoring this aspect of liver function is of particular interest in the present invention.

According to a further aspect of the invention there is provided a fusion protein comprising:
(i) a biologically active fragment of CSF-1 or a homolog or a variant or a derivative thereof; and
(ii) a biologically active antibody fragment.

Preferably, for the purpose of human therapy the biologically active fragment of CSF-1 is residues 33-182 of human CSF-1 (SEQ ID NO:5) or a biologically active portion thereof, or the biological equivalent fragment of CSF-1 from any mammalian species.

The biologically active fragment of CSF-1 may be native or it may be recombinant.

Preferably, the antibody is an immunoglobulin selected from the group comprising IgA, IgD, IgE, IgG and IgM more preferably it is IgG.

Preferably, the antibody fragment is selected from the group comprising F(ab′)2, Fab′, Fab, Fv, Fc and rIgG and more preferably it is an FC fragment.

Preferably, the biologically active fragment of CSF-1 or a homolog or a variant or derivative thereof and the biologically active antibody fragment of the fusion protein are covalently linked directly or through a linker moiety.

According to a further aspect of the invention there is provided a nucleic acid encoding the fusion protein.

According to a yet further aspect of the invention there is provided a vector comprising the isolated nucleic acid of the invention.

According to a yet further aspect of the invention there is provided a host cell comprising the vector of the invention.

According to a yet further aspect of the invention there is provided a method of making the fusion protein of the first aspect of the invention, the method comprising:
(i) culturing the host cell of the present invention; and
(ii) collecting the fusion protein from said culture.

According to a yet further aspect of the invention there is provided a composition comprising:
(a) at least one fusion protein comprising (i) a biologically active fragment of CSF-1 or a homolog or a variant or a derivative thereof; and (ii) a biologically active antibody fragment; and
(b) a pharmaceutically acceptable carrier, excipient or diluents.

In alternative embodiments the composition may include the nucleic acid or vector of the present invention.

According to a further aspect of the invention there is provided use of a fusion protein comprising:
(i) a biologically active fragment of CSF-1 or a homolog or a variant or a derivative thereof; and
(ii) a biologically active antibody fragment
   for enhancing liver regeneration and/or restoring liver function and/or modulating liver homeostasis.

In the surgical setting, surgical removal of the region of the liver containing the liver cancer is the mainstay of curative management. This can risk postoperative liver failure, especially if the patient has a background of chronic liver disease. In the context of liver transplantation, liver failure may ensue if the transplanted organ is insufficient to meet the demands of the recipient. Treatment with a therapy to enhance regeneration could be applied before, during or following surgery.

According to a further aspect of the invention there is provided use of the fusion protein or the nucleic acid or vector of the present invention for the manufacture of a medicament for enhancing liver regeneration and/or restoring liver function and/or modulating liver homeostasis.

According to a yet further aspect of the invention there is provided a method of treatment for an individual suffering from liver cancer and who is to undergoing surgery, the method comprising administering the fusion protein or the nucleic acid or vector of the present invention before, during or after the surgical procedure.

According to a yet further aspect of the invention there is provided a method of treatment for an individual who is undergoing liver transplant surgery, the method comprising administering the fusion protein or the nucleic acid or vector of the present invention before, during or after the surgical procedure.

According to a yet further aspect of the invention there is provided a kit comprising one or more containers having pharmaceutical dosage units comprising an effective amount of the fusion protein or nucleic acid or vector of the present invention, wherein the container is packaged with optional instructions for the use thereof.

The various aspects of the present invention provide compositions and methods of enhancing regeneration or restoring function of an injured liver in humans and other mammalian species

Features ascribed to any aspect of the invention are applicable mutatis mutandis to all other aspects of the invention.

### BRIEF DESCRIPTION OF THE DRAWINGS

Embodiments of the invention are further described hereinafter, by way of non-limiting example, with reference to the accompanying drawings, in which:

FIG. 1 shows a Kaplein Meir of survival in three injury models and chart shows percentage weight change following intervention and treatment. ***p<0.001 Mann Whitney U. [Solid line=treatment with fc-CSF1; broken line=PBS control]

FIG. 2 is a bar chart showing Liver weight/Body weight ratio expressed as a percentage and hepatocyte proliferation expressed as Ki67 positive hepatocytes per high-powered field. *p<0.05; **p<0.01; ***p<0.001 Mann Whitney U. [Treatment=fc-CSF1; Control=PBS]

FIG. 3 shows the effect of CSF-1 administered as described above on body weight. FIG. 3A compares CSF-1 (1 mg/kg) with Fc-CSF-1 (1 mg/kg). The unmodified protein has no effect at this dose, where Fc-CSF-1 clearly increased total body weight. FIG. 3B shows a dose response curve, demonstrating detectable activity at 0.1 mg/kg of Fc-CSF-1.

FIG. 3C shows the effect of 1 mg/kg dose is confirmed in a larger experimental series. The animals in this series are analysed further in subsequent slides

FIG. 4A shows the effect of CSF-1 (1 mg/kg) and Fc-CSF-1 (1 mg/kg) on mouse spleen weight, FIG. 4B shows the effect of CSF-1 (1 mg/kg) and Fc-CSF-1 (1 mg/kg) on mouse liver weight.

FIG. 5 shows the effect of Fc-CSF-1 on the numbers of macrophages in the spleen, detected with the csf1r-EGFP reporter, FIG. 5A is the control and FIG. 5B shows the treated sample.

FIG. 6A shows a dose response curve for FIGS. 5A and 5B, FIG. 6A shows a dose response curve based upon immunohistochemical localisation of the macrophage-specific F4/80 antigen.

FIG. 7 shows immunostaining for the macrophage-specific F4/80 antigen in mice. FIG. 7A shows PBS treated control liver; FIG. 7B shows the liver of a mouse treated with Fc-CSF-1, FIG. 7C shows PBS treated control spleen FIG. 7D shows the spleen of a mouse treated with Fc-CSF-1.

FIG. 8A shows a PBS treated control mouse liver with immunostaining for proliferating cell nuclear antigen (PCNA), FIG. 8B shows a mouse liver following treatment with Fc-CSF-1.

FIG. 9 shows the impact of pharmacokinetics of CSF-1 administered to weaner pigs. FIG. 9A shows the clearance of unmodified CSF-1, FIGS. 9B and 9C show the clearance of 1.2 mg/kg of Fc-CSF-1 when administered intravenously and subcutaneously respectively.

FIG. 10 shows the blood effects in weaner pigs administered 0.5 mg/Kg×6; FIG. 10A shows the total white blood count, FIG. 10B shows the monocyte count, FIG. 100 shows the lymphocyte count and FIG. 10D shows the neutophil count.

FIG. 11 the dose response curves of blood effects in weaner administered 0.5 mg/Kg×6; FIG. 11A shows the total white blood count, FIG. 11B shows the monocyte count, FIG. 11C shows the lymphocyte count and FIG. 11D shows the neutrophil count.

FIG. 12 shows the effect on organ weights in weaner pigs administered 0.12 mg/Kg×3. FIG. 12A shows the effect on liver weight, FIG. 12B shows the effect on spleen weight, FIG. 11C shows the effect on lung weight and FIG. 11D shows the effect on kidney weight.

FIG. 13A shows serum CSF1 level in patients at admission in patients who survived or died/underwent liver transplantation with paracetamol induced liver failure. FIG. 13B shows serum levels of a subset of patients who subsequently died or survived. FIG. 13 C shows receiver operating characteristic curve analysis assessing the potential of admission CSF1 to serve as a biomarker for survival without transplantation following paracetamol overdose.

FIG. 14A shows hepatic CSF1 gene expression following paracetamol intoxication and serum CSF1 level. FIG. 14 B shows liver to bodyweight ratio and hepatocyte proliferation assessed by Ki67 immunohistochemistry at Day 3 following paracetamol intoxication. FIG. 14C shows serum analysis at Day 3 post paracetamol intoxication comparing control and CSF1 receptor inhibition.

FIG. 15A shows mean liver weight to body weight ratio and hepatocyte proliferation (ki67 immunohistochemistry) in mice following paracetamol intoxication comparing CSF1-Fc (solid line) or control (dotted line) administration. FIG. 15B shows serum parameters post paracetamol intoxication. (n=8/group).

FIG. 16A shows hepatic CSF1 gene expression following ⅔ partial hepatectomy and serum CSF1 level. FIG. 16B shows liver to bodyweight ratio and hepatocyte proliferation assessed by Ki67 immunohistochemistry at Day 2 following ⅔ partial hepatectomy with CSF1 receptor inhibition (GW2580) or control. FIG. 16C shows serum analysis at Day 2 post paracetamol intoxication comparing control and CSF1 receptor inhibition with GW2580. (n=8 per group).

FIG. 17A shows mean liver weight to body weight ratio and hepatocyte proliferation (ki67 immunohistochemistry) in mice following ⅔ partial hepatectomy comparing CSF1-Fc (solid line) or control (dotted line) administration. FIG. 17B shows serum parameters post paracetamol intoxication. (n=8/group). FIG. 17C shows relative gene expression of the proregenerative cytokines II6 and oncostatin M (OSM) and also a growth factor activator urokinase receptor (UR) with blockade of CSF1 receptor (GW2580) and administration of CSF1-Fc versus controls.

FIG. 18A shows Kaplan Meir plot showing trend to survival (p=0.07) and increased body weight postoperatively with CSF1-Fc treatment following partial hepatectomy in the chronically injured liver (solid line=CSF1-Fc, dotted line=control; n=8/group at Day 4 and Day 7). FIG. 18B shows mean liver weight to body weight ratio, hepatocyte proliferation (ki67 immunohistochemistry) and fibrosis quantification via Sirius red quantification. FIG. 18C shows serum parameters.

FIG. 19 shows A) gene expression relative to mean of control group (MARCO: macrophage receptor with collagenous structure; MSR1: macrophage scavenger receptor 1) (white=control; shaded=CSF1-Fc); B) Bead clearance assay showing flow plot overlay gated on fluorescent beads from 1-15 minutes following intravascular injection (dotted line=control; solid line=CSF1-Fc; grey line=uninjured untreated mouse); C) ex vivo fluorescence organs 15 minutes following intravascular injection of fluorescent beads. (n=6 per group)

FIG. 20 shows A) gene expression relative to mean of control group (MARCO: macrophage receptor with collagenous structure; MSR1: macrophage scavenger receptor 1) [white=control; shaded=CSF1-Fc]; B) bead clearance assay; C) ex vivo fluorescence organs 15 minutes following intravascular injection of fluorescent beads.

FIG. 21 shows A) Serum CSF1 level of 55 patients undergoing partial hepatectomy taken preoperatively and on postoperative day 1 and postoperative day 3. B) Cohort segregated according to extent of liver resection. Two way ANOVA with post hoc analysis showing significant increase in CSF1 level in patients who had more than 5 segments resected compared to patients who had less than 3 segments resected. C) Patients who developed postoperative liver failure shown in dots compared to rest of the cohort (median and range).

### DETAILED DESCRIPTION

The terms “M-CSF”, “macrophage colony stimulating factor”, “CSF-1”, “CSF1”, “colony stimulating factor1” and “colony stimulating factor-1” are used interchangeably herein.

By the term “supplementation”, “supplement” or “supplementing”, it is intended that CSF-1 is administered to an individual in an additional or extra amount in excess of the level that the individual already has of functioning CSF-1.

By the terms “treat,” “treating” or “treatment of,” it is intended that the severity of the disorder or the symptoms of the disorder are reduced, or the disorder is partially or entirely eliminated, as compared to that which would occur in the absence of treatment. Treatment does not require the achievement of a complete cure of the disorder.

By the terms “restore,” “restoring” or “restoration of,” it is intended that the severity of the disorder or the symptoms of the disorder are reduced, or the disorder is partially or entirely eliminated, as compared to that which would occur in the absence of treatment. Treatment does not require the achievement of a complete cure of the disorder.

A “therapeutically effective” or “effective” amount is intended to designate a dose that causes a relief of symptoms of a disease or disorder as noted through clinical testing and evaluation, patient observation, and/or the like. “Effective amount” or “effective” can further designate a dose that causes a detectable change in biological or chemical activity. The detectable changes may be detected and/or further quantified by one skilled in the art for the relevant mechanism or process. Moreover, “effective amount” or “effective” can designate an amount that maintains a desired physiological state, i.e., reduces or prevents significant decline and/or promotes improvement in the condition of interest. As is generally understood in the art, the dosage will vary depending on the administration routes, symptoms and body weight of the patient but also depending upon the compound being administered.

Conditions which can be treated in the present invention include liver damage or hepatitis as the result of physical trauma, adverse action of pharmaceuticals or toxic chemicals, infection, autoimmunity, ischaemia, alcohol induced liver damage, or any other cause of liver damage. Liver injury is commonly caused by physical trauma such as road traffic accidents, falls, assault or the like. Paracetamol (acetaminophen) overdose is a relatively common cause of pharmaceutical-induced liver damage, but liver damage can also be caused by many other pharmaceuticals, e.g. methotrexate, statins, niacin, amiodarone, chemotherapy agents, and some antibiotics. Alcohol-induced liver disease is a very widespread cause of liver damage. Infections that cause liver damage include, amongst others, hepatitis A, B or C viral infections. While it is probable that CSF1 treatment will not be appropriate in all cases of liver damage, in many cases it may have a beneficial effect.

By the term “Fc” it is intended to refer to a region of an antibody molecule that binds to antibody receptors on the surface of cells such as macrophages and mast cells, and to complement protein. Fc (50,000 daltons) fragments contain the CH2 and CH3 region and part of the hinge region held together by one or more disulfides and non-covalent interactions. Fc and Fc5μ fragments are produced from fragmentation of IgG and IgM, respectively. The term Fc is derived from the ability of these antibody fragments to crystallize. Fc fragments are generated entirely from the heavy chain constant region of an immunoglobulin. The Fc fragment cannot bind antigen, but it is responsible for the effector functions of antibodies, such as complement fixation.

“Polypeptide” refers to a polymer of amino acids (dipeptide or greater) linked through peptide bonds. Thus, the term “polypeptide” includes proteins, oligopeptides, protein fragments, protein analogs and the like. The term “polypeptide” contemplates polypeptides as defined above that are encoded by nucleic acids, are recombinantly produced, are isolated from an appropriate source, or are synthesized.

As used herein, a “functional” polypeptide is one that retains at least one biological activity normally associated with that polypeptide. Preferably, a “functional” polypeptide retains all of the activities possessed by the unmodified peptide. By “retains” biological activity, it is meant that the polypeptide retains at least about 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits essentially no detectable biological activity normally associated with the polypeptide (e.g., at most, only an insignificant amount, e.g., less than about 10% or even 5%).

“Fusion protein” as used herein, refers to a protein produced when two heterologous nucleotide sequences or fragments thereof coding for two (or more) different polypeptides, or fragments thereof, are fused together in the correct translational reading frame. The two or more different polypeptides, or fragments thereof, include those not found fused together in nature and/or include naturally occurring mutants.

As used herein, a “fragment” is one that substantially retains at least one biological activity normally associated with that protein or polypeptide. In particular embodiments, the “fragment” substantially retains all of the activities possessed by the unmodified protein. By “substantially retains” biological activity, it is meant that the protein retains at least about 50%, 60%, 75%, 85%, 90%, 95%, 97%, 98%, 99%, or more, of the biological activity of the native protein (and can even have a higher level of activity than the native protein).

A “recombinant polypeptide” is one that is produced from a recombinant nucleic acid.

An “isolated” polypeptide means a polypeptide that is separated or substantially free from at least some of the other components of the naturally occurring organism or virus, for example, the cell or viral structural components or other polypeptides or nucleic acids commonly found associated with the polypeptide. As used herein, the “isolated” polypeptide is at least about 25%, 50%, 60%, 70%, 75%, 80%, 85%, 90%, 95%, 97%, 98%, 99% or more pure (w/w).

The term “derivative” is to be understood to refer to any molecule that is derived (substantially derived) or obtained (substantially obtained) from CSF-1, but retains similarity, or substantial similarity, in biological function of CSF-1. In certain aspects, the biological function is the ability to promote liver organ development. A derivative may, for instance, be provided as a result of cleavage of CSF-1 to produce biologically-active fragments, cyclisation, bioconjugation and/or coupling with one or more additional moieties that improve, for example, solubility, stability or biological half-life, or which act as a label for subsequent detection or the like. A derivative may also result from post-translational or post-synthesis modification such as the attachment of carbohydrate moieties, or chemical reactions(s) resulting in structural modification(s) such as alkylation or acetylation of an amino acid(s) or other changes involving the formation of chemical bonds. In a particularly preferred embodiment of a derivative suitable for use in the present invention, the derivative is the mature domain of CSF-1. In another preferred embodiment of a derivative suitable for use in the methods disclosed herein, the derivative is a biologically active, C-terminal fragment of CSF-1 (e.g. a CSF-1 fragment comprising the C-terminal amino acids 1 to 150 of the 536 amino acid protein). Further embodiments of a derivative of CSF-1 include CSF-1 comprising chemically modified side chains (e.g. pegylation of lysyl ε-amino groups), C- and/or N-termini (e.g. acylation of the N-terminal with acetic anhydride), or linked to various carriers (e.g. human serum albumin or histidine (His6) tag).

As generally used herein, a “homolog” shares a definable nucleotide or amino acid sequence relationship with another nucleic acid or polypeptide as the case may be. A “protein homolog” preferably shares at least 70% or 80% sequence identity, more preferably at least 85%, 90% and even more preferably at least 95%, 96%, 97%, 98% or 99% sequence identity with the amino acid sequences of polypeptides as described herein. Homologs of CSF may also be used in accordance with the invention. Such CSF homologs would preferably be characterized by biological activity about the same or greater than that of a CSF protein having a high or substantial biological activity.

As used herein, “variant” proteins are proteins in which one or more amino acids have been replaced by different amino acids. Protein variants of CSF that retain biological activity of native or wild type CSF may be used in accordance with the invention. It is well understood in the art that some amino acids may be changed to others with broadly similar properties without changing the nature of the activity of the polypeptide (conservative substitutions). Generally, the substitutions which are likely to produce the greatest changes in a polypeptide's properties are those in which (a) a hydrophilic residue (e.g., Ser or Thr) is substituted for, or by, a hydrophobic residue (e.g. Leu, lie, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a bulky side chain (e.g., Phe or Trp) is substituted for, or by, one having a smaller side chain (e.g., Ala, Ser) or no side chain (e.g., Gly).

Embodiments of the present invention further provide an isolated nucleic acid (e.g., an “isolated DNA” or an “isolated vector genome”) that encodes the fusion protein described herein. The nucleic acid is separated or substantially free from at least some of the other components of the naturally occurring organism or virus, such as for example, the cell or viral structural components or other polypeptides or nucleic acids commonly found associated with the nucleic acid. The coding sequence for a polypeptide constituting the active agents of the present invention is transcribed, and optionally, translated. According to embodiments of the present invention, transcription and translation of the coding sequence will result in production of a fusion protein described.

It will be appreciated by those skilled in the art that there can be variability in the nucleic acids that encode the fusion polypeptides of the present invention due to the degeneracy of the genetic code. Further variation in the nucleic acid sequence can be introduced by the presence (or absence) of non-translated sequences, such as intronic sequences and 5′ and 3′ untranslated sequences. Moreover, the isolated nucleic acids of the invention encompass those nucleic acids encoding fusion proteins that have at least about 60%, 70%, 80%, 90%, 95%, 97%, 98% or higher amino acid sequence similarity with the polypeptide sequences specifically disclosed herein or to those known sequences corresponding to proteins included in aspects of the present invention (or fragments thereof) and further encode functional fusion proteins as defined herein

Isolated nucleic acids of this invention include RNA, DNA (including cDNAs) and chimeras thereof. The isolated nucleic acids can further comprise modified nucleotides or nucleotide analogs.

The isolated nucleic acids encoding the polypeptides of the invention can be associated with appropriate expression control sequences, e.g., transcription/translation control signals and polyadenylation signals.

It will be appreciated that a variety of promoter/enhancer elements can be used depending on the level and tissue-specific expression desired. The promoter can be constitutive or inducible (e.g., the metalothionein promoter or a hormone inducible promoter), depending on the pattern of expression desired. The promoter can be native or foreign and can be a natural or a synthetic sequence. By foreign, it is intended that the transcriptional initiation region is not found in the wild-type host into which the transcriptional initiation region is introduced. The promoter is chosen so that it will function in the target cell(s) of interest.

The present invention further provides methods of making fusion proteins described herein. Methods of making fusion proteins are well understood in the art. Such methods include growing a host cell including a vector that includes nucleic acids encoding the fusion protein under conditions appropriate for expression and subsequent isolation of the fusion protein. Accordingly, the isolated nucleic acids encoding a polypeptide constituting the fusion protein of the invention can be incorporated into a vector, e.g., for the purposes of cloning or other laboratory manipulations, recombinant protein production, or gene delivery. Exemplary vectors include bacterial artificial chromosomes, cosmids, yeast artificial chromosomes, phage, plasmids, lipid vectors and viral vectors (described in more detail below).

In particular embodiments, the isolated nucleic acid is incorporated into an expression vector. In further embodiments of the present invention, the vector including the isolated nucleic acids described herein are included in a host cell. Expression vectors compatible with various host cells are well known in the art and contain suitable elements for transcription and translation of nucleic acids. Typically, an expression vector contains an “expression cassette,” which includes, in the 5′ to 3′ direction, a promoter, a coding sequence encoding a polypeptide of the invention or active fragment thereof operatively associated with the promoter, and, optionally, a termination sequence including a stop signal for RNA polymerase and a polyadenylation signal for polyadenylase.

In addition to the regulatory control sequences discussed above, the recombinant expression vector can contain additional nucleotide sequences. For example, the recombinant expression vector can encode a selectable marker gene to identify host cells that have incorporated the vector and/or may comprise another heterologous sequence of interest.

Vectors can be introduced into prokaryotic or eukaryotic cells via conventional transformation or transfection techniques. As used herein, the terms “transformation” and “transfection” refer to a variety of art-recognized techniques for introducing foreign nucleic acids (e.g., DNA) into a host cell, including calcium phosphate or calcium chloride co-precipitation, DEAE-dextran-mediated transfection, lipofection, electroporation, microinjection, DNA-loaded liposomes, lipofectamine-DNA complexes, cell sonication, gene bombardment using high velocity microprojectiles, and viral-mediated transfection.

In terms of administration, the most suitable route in any given case will depend on the nature and severity of the liver condition being treated and on the fusion protein, viral vector, nucleic acid or pharmaceutical formulation being administered.

The fusion proteins, viral vectors and nucleic acids (e.g., DNA and/or RNA) of the invention can be formulated for administration in a pharmaceutical carrier in accordance with known techniques. See, e.g., Remington, The Science And Practice of Pharmacy (9th Ed. 1995). In the manufacture of a pharmaceutical formulation according to the invention, the fusion protein, viral vector or nucleic acid is typically admixed with, inter alia, an acceptable carrier. The carrier can be a solid or a liquid, or both, and is optionally formulated as a unit-dose formulation, which can be prepared by any of the well-known techniques of pharmacy.

The carriers and additives used for such pharmaceutical compositions can take a variety of forms depending on the anticipated mode of administration. Thus, compositions for oral administration may be, for example, solid preparations such as tablets, sugar-coated tablets, hard capsules, soft capsules, granules, powders and the like, with suitable carriers and additives being starches, sugars, binders, diluents, granulating agents, lubricants, disintegrating agents and the like. Because of their ease of use and higher patient compliance, tablets and capsules represent the most advantageous oral dosage forms for many medical conditions.

Similarly, compositions for liquid preparations include solutions, emulsions, dispersions, suspensions, syrups, elixirs, and the like with suitable carriers and additives being water, alcohols, oils, glycols, preservatives, flavoring agents, coloring agents, suspending agents, and the like.

In the case of a solution, it can be lyophilized to a powder and then reconstituted immediately prior to use. For dispersions and suspensions, appropriate carriers and additives include aqueous gums, celluloses, silicates or oils.

For injection, the carrier is typically a liquid, such as sterile pyrogen-free water, pyrogen-free phosphate-buffered saline solution, bacteriostatic water, or Cremophor EL[R] (BASF, Parsippany, N.J.), parenterally acceptable oil including polyethylene glycol, polyvinyl pyrrolidone, lecithin, arachis oil or sesame oil, with other additives for aiding solubility or preservation may also be included. For other methods of administration, the carrier can be either solid or liquid.

For oral administration, the fusion protein, viral vector or nucleic acid can be administered in solid dosage forms, such as capsules, tablets, and powders, or in liquid dosage forms, such as elixirs, syrups, and suspensions. The fusion protein, viral vector or nucleic acid can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations of the present invention suitable for parenteral administration can include sterile aqueous and non-aqueous injection solutions of the fusion protein, viral vector or nucleic acid, which preparations are generally isotonic with the blood of the intended recipient. These preparations can contain anti-oxidants, buffers, bacteriostats and solutes, which render the formulation isotonic with the blood of the intended recipient. Aqueous and non-aqueous sterile suspensions can include suspending agents and thickening agents. The formulations can be presented in unit\dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets. For example, in one aspect of the present invention, there is provided an injectable, stable, sterile composition including a fusion protein, viral vector or nucleic acid of the invention, in a unit dosage form in a sealed container. Optionally, the composition is provided in the form of a lyophilizate, which is capable of being reconstituted with a suitable pharmaceutically acceptable carrier to form a liquid composition suitable for injection thereof into a subject.

In particular embodiments of the invention, administration is by subcutaneous or intradermal administration. Subcutaneous and intradermal administration can be by any method known in the art including, but not limited to, injection, gene gun, powderject device, bioject device, microenhancer array, microneedles, and scarification (i.e., abrading the surface and then applying a solution including the fusion protein, viral vector or nucleic acid).

In other embodiments, the fusion protein, viral vector or nucleic acid is administered intramuscularly, for example, by intramuscular injection or by local administration.

Nucleic acids (e.g., DNA and/or RNA) can also be delivered in association with liposomes, such as lecithin liposomes or other liposomes known in the art (for example, as described in WO 93/24640) and may further be associated with an adjuvant. Liposomes including cationic lipids interact spontaneously and rapidly with polyanions, such as DNA and RNA, resulting in liposome/nucleic acid complexes that capture up to 100% of the polynucleotide. In addition, the polycationic complexes fuse with cell membranes, resulting in an intracellular delivery of polynucleotide that bypasses the degradative enzymes of the lysosomal compartment. PCT publication WO 94/27435 describes compositions for genetic immunization including cationic lipids and polynucleotides. Agents that assist in the cellular uptake of nucleic acid, such as calcium ions, viral proteins and other transfection facilitating agents, may be included.

According to the present invention, methods of this invention include administering an effective amount of a composition of the present invention as described above to the subject. The effective amount of the composition, the use of which is in the scope of present invention, will vary somewhat from subject to subject, and will depend upon factors such as the age and condition of the subject and the route of delivery. Such dosages can be determined in accordance with routine pharmacological procedures known to those skilled in the art. For example, the active agents of the present invention can be administered to the subject in an amount ranging from a lower limit from about 0.01, 0.05, 0.10, 0.50, 1.0, 5.0, or 10% to an upper limit ranging from about 10, 20, 30, 40, 50, 60, 70, 80, 90, 95, 96, 97, 98, 99, or 100% by weight of the composition. In some embodiments, the active agents include from about 0.05 to about 95% by weight of the composition. In other embodiments, the active agents include from about 0.05 to about 60% by weight of the composition. In still other embodiments, the active agents include from about 0.05 to about 10% by weight of the composition.

### Cloning and Expression of the Pig Fc-Fusion

The sequence corresponding to the active fragment of porcine CSF-1 (SENCSHMIGDGHLKVLQQLIDSQMETSCQIAFEFVDQEQLTDPVCYLKKAFLQVQDILDE TMRFRDNTPNANVIVQLQELSLRLNSCFTKDYEEQDKACVRTFYETPLQLLEKIKNVFNET KNLLKKDWNIFSKNCNNSFAKCSSQHERQPEGR) (SEQ ID NO:1) was linked to the hinge-CH3 region of the porcine IgG1a sequence (GTKTKPPCPICPGCEVA GPSVFIFPPKPKDTLMISQTPEVTCVVVDVSKEHAEVQFSVVYVDGVEVHTAETRPKEEQF NSTYRVVSVLPIQHQDWLKGKEFKCKVNNVDLPAPITRTISKAIGQSREPQVYTLPPPAEE LSRSKVTVTCLVIGFYPPDIHVEWKSNGQPEPEGNYRTTPPQQDVDGTFFLYSKLAVDKA RWDHGETFECAVMHEALHNHYTQKSISKTQGK) (SEQ ID NO:2). This entire region was codon optimized for mammalian expression by GeneArt (Invitrogen, CA, USA) and cloned into the expression plasmid pS00524 using HindIII and NotI restriction sites engineered into the 5′ and 3′ ends respectively. The resulting plasmid was sequenced to ensure ORF integrity and protein was expressed from transfected HEK293F or CHO cells.

### Isolation of Pig CSF-1:Fc Fusion

Porcine CSF-1 Fc fusion protein was isolated using Protein A affinity chromatography. Briefly, conditioned medium from cell culture was clarified and loaded onto Protein A Sepharose that was equilibrated with PBS. Following loading the column was washed with 2 BV of PBS and 2 BV of 35 mM Na Acetate pH 5.5. Protein was eluted using a step gradient of 80% B Buffer (35 mM Acetic acid, no pH adjustment), 85% B buffer and 100% B buffer. The 80 and 85% B fractions were pooled based on lack of aggregated protein (analytical SEC) and the 100% B fraction was not included. Pooled protein was pH adjusted to 7.2 and dialyzed against PBS.

### Porcine CSF-1 Fc-Fusion Quantitation in Blood Plasma by ELISA

Porcine CSF-1 Fc-fusion plasma levels were detected using an in-house developed conventional sandwich ELISA utilizing commercially available antibodies. Capture antibody was Abcam ab9693 (0.3 μg/mL) and detection antibody was Rabbit anti-pig IgG (Fc) biotinylated Alpha Diagnostic 90440 (1:5000 dilution). Standard protein was generated and purified in-house (lot 2/24/11 JAS). Standards were added to each plate along with the samples resulting in an 11 point standard range of 2700 ng/mL to 0.046 pg/mL. This allowed for quantitation of each sample to a standard curve on every assay plate. Assay detection was done using Pierce High Sensitivity Streptavidin-HRP (1:10,000 dilution) and TMB Microwell Peroxidase Substrate System solution (KPL).

### Pig PK

Weaner age barrows (<14 kg) were assigned to three treatment groups receiving a single intravenous (IV) or subcutaneous (SC) dose as follows. Three pigs received 0.5 mg/kg CSF-1 non-fusion dosed SC. Two pigs received 1.2 mg/kg CSF-1:Fc fusion dosed IV. Two pigs received 1.2 mg/kg CSF-1:Fc dosed SC. One ml plasma samples were obtained via the V. jugularis in EDTA anticoagulant tubes and placed on ice until centrifuged. The plasma was transferred to sterile tubes stored at ≦−10° C. until analysis. Serial plasma samples were obtained from each animal at pre-dose and 5 minutes, 30 minutes, 1 hour, 2 hours, 4 hours, 6 hours, 8, hours, 24 hours, 48 hours, and 72 hours post-dose. CSF-1 and CSF-1:Fc fusion protein levels were quantitated in plasma using ELISA assays.

### MTT Cell Viability Assay

Stable Ba/F3 cells expressing porcine CSF-1R were maintained in culture with complete RPMI supplemented with either 10⁴ Units/ml rh-CSF-1 or 10% IL-3 conditioned medium prior to MTT assay. 2×10⁴ cells/well (Ba/F3 cells and Ba/F3 transfectants), or 5×10⁴ cells/well (pig BMM) of a 96 well plate were plated in triplicate or quadruplicate and appropriate treatment (serial dilutions of rh-CSF-1 or porcine Fc CSF-1 were added to make a total volume of 100 μl per well. Cells were incubated for 48 hours at 37° C. with 5% CO₂. For Ba/F3 cells, 10 μl of MTT (Sigma Aldrich M5655) stock solution (5 mg/ml) was added directly to each well to achieve a final concentration of 0.5 mg/ml and incubated at 37° C. for 3 hours prior to solubilisation overnight. For adherent mouse BMM cells, culture medium was replaced with 50 μl of 1 mg/ml MTT solution and incubated for 1 hour at 37° C. MTT solution was removed and tetrazolium salt solubilised with 100 μl of solubilisation agent (0.1M HCL, 10% Triton x-100 and isopropanol) followed by incubation at 37° C. with 5% CO2 for 10 minutes. Plates were read at 570 nm with reference wavelength of 405 nm.

### Mice Studies

Forty-eight male C57Bl6 mice aged 10-12 weeks underwent either ⅔ partial hepatectomy, paracetamol intoxication or chronic liver injury plus ⅔ partial hepatectomy. ⅔ partial hepatectomy was performed by ligating the left lobe and left and right median lobes. Paracetamol intoxication was performed by intraperitoneal administration of 350 mg/kg paracetamol dissolved in phosphate buffered saline (PBS). Chronic liver injury plus ⅔ partial hepatectomy was performed by eight weeks intraperitoneal carbon tetrachloride administration 1 mcl/g twice weekly dissolved in olive oil followed by ⅔ partial hepatectomy as described above. The treatment group (n=8 per injury model) received 0.75 mcg/g FC-CSF1 administered subcutaneously immediately following either partial hepatectomy or paracetamol intoxication and subsequently every 24 hours for three further doses. Control mice (n=8 per injury model) received subcutaneous PBS of appropriate volume. All mice were culled on day 4 following injury (partial hepatectomy or paracetamol intoxication).

Following a midline laparotomy the liver was excised and weighed. Liver weight to body weight ratio was calculated and expressed as a percentage. Livers were fixed in 4% formalin overnight then transferred to 70% ethanol. Livers were then embedded in paraffin blocks and 4 μm sections cut. Immunohistochemistry for Ki67 (a marker of cellular proliferation expressed throughout the cell cycle) was performed following heat mediated antigen retrieval in Tris/EDTA solution at pH9. Hepatocyte proliferation was quantified by counting ki67 positive hepatocytes in 20 high powered fields (400×) per animal and the mean calculated.

### Example 1

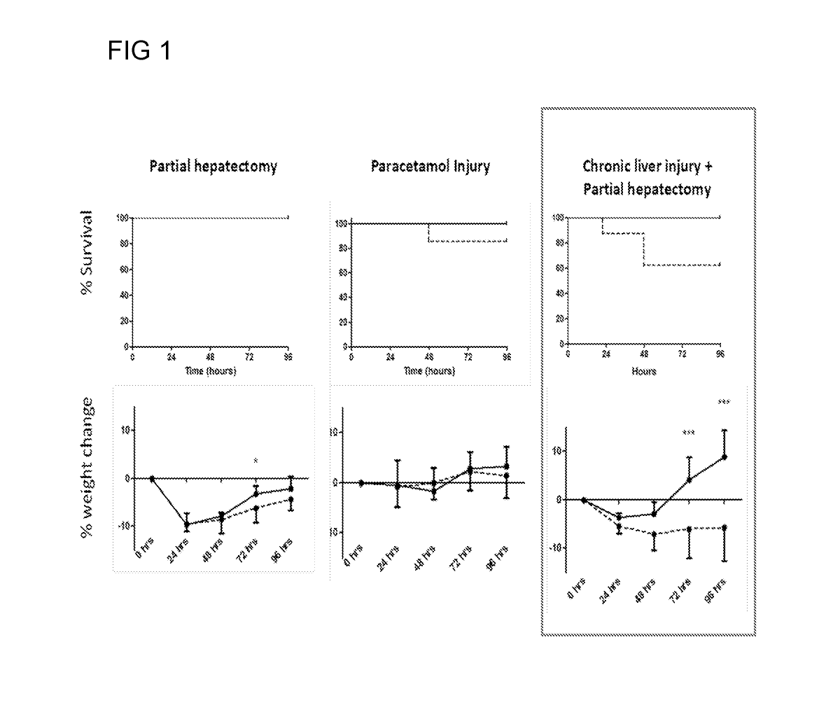

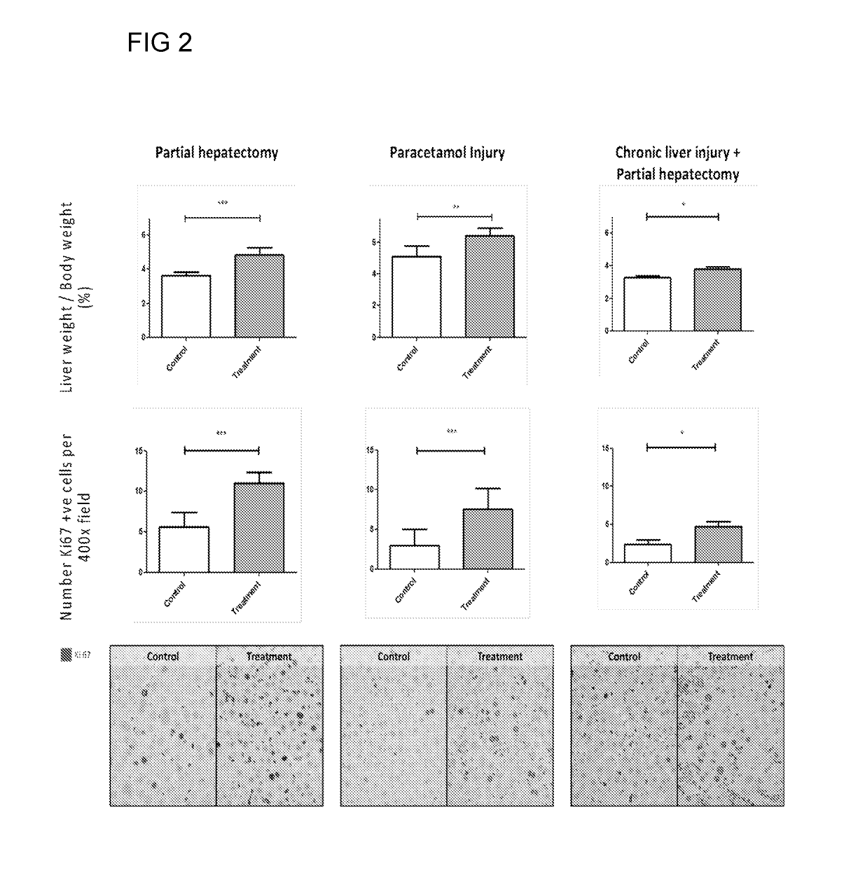

The effects of Fc-CSF1 on liver regeneration in murine models of acute liver injury (partial hepatectomy; paracetamol intoxication) and acute-on-chronic liver injury (chronic liver injury plus partial hepatectomy) were studied. The treatment group (n=8 per injury model) received 0.5 mcg/g Fc-CSF1 administered subcutaneously immediately following either partial hepatectomy or paracetamol intoxication and subsequently every 24 hours for three further doses. Control mice (n=8 per injury model) received subcutaneous PBS of appropriate volume. Interventions and treatments were well tolerated. In the most severe injury model (chronic liver injury with ⅔ partial hepatectomy) there was a significant increase in mouse weight (p<0.001 at day 4) and a trend to improved survival (p=0.08) (FIG. 1). Others findings included enhanced regenerative parameters of both liver weight and hepatocyte proliferation across the injury models (FIG. 2). The results of these studies demonstrate that administration of Fc-CSF1 can enhance the regenerative response across a range of hepatic injury models. In the most severe model of hepatic injury (chronic liver injury plus partial hepatectomy) there was a trend to improved survival and a significant body weight increase indicating improved postoperative course. Fc-CSF1 was found to have a growth promoting effect on hepatic weights and hepatocyte proliferation in all injury models. While there is redundancy in many of the pathways leading to effective liver regeneration it appears that CSF1 is critical to achieve optimal recovery and the present studies have shown that supplementation of this factor can further boost regeneration. It is envisaged these findings will translate to improved outcomes in the management of liver failure in the clinical setting.

### Example 2

Mice were injected with Fc-CSF-1 subcutaneously on each of 4 days and sacrificed on the 5^{th} day. The mice were csf1r-EGFP (MacGreen) mice on the C57Bl/6 background. Tissue processing and immunohistochemistry were carried out as described in (Alikhan et al Am J.Pathol. 179, 1243-1256, 2011 and Macdonald et al Blood. 116, 3955-3963, 2010). A comparison of the effect of recombinant pig CSF-1 or Fc-CSF-1 on the proliferation of mouse bone marrow cells or the Ba/F3 CS1R reporter cell line using the assay described in Gow et al Cytokine. 60, 793-805, 2012) showed that there was no difference in biological activity (data not shown), demonstrating that additional of the Fc component to the C terminus of CSF-1 does not interfere with binding to the receptor.

### Example 3

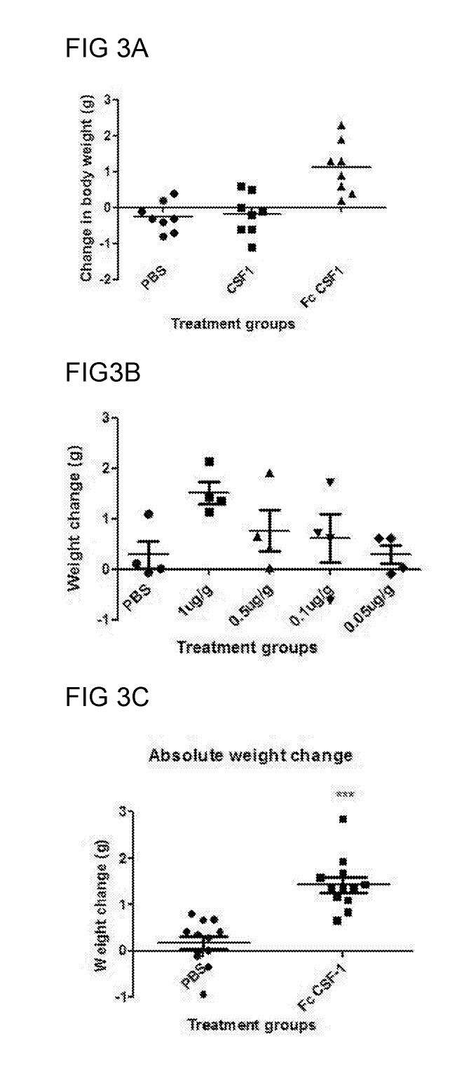

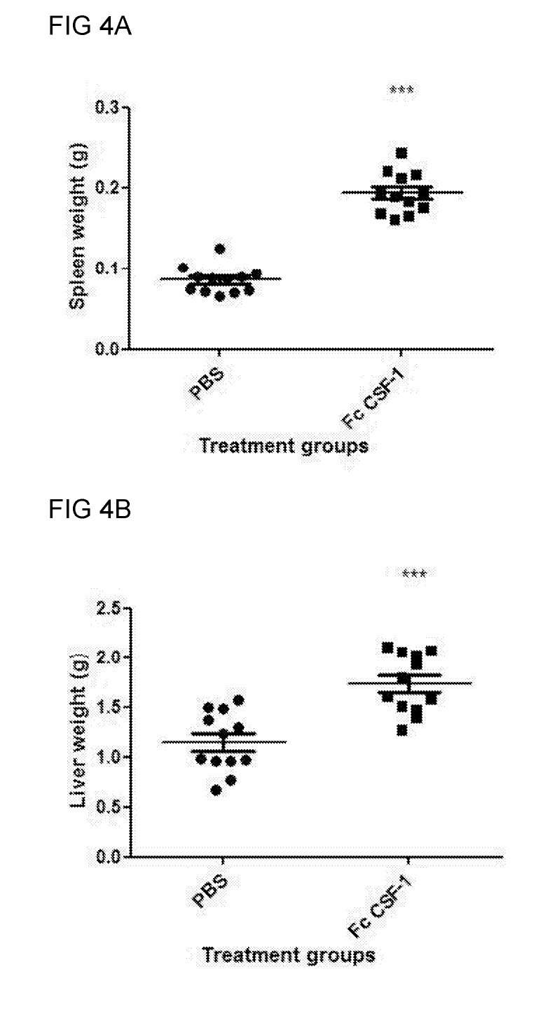

The effect of administered CFS-1 on body weight was assessed. FIG. 3A compares CSF-1 (1 mg/kg) with Fc-CSF-1 (1 mg/kg). The unmodified protein has no effect at this dose, where Fc-CSF-1 clearly increase total body weight. FIG. 3B shows a dose response curve, demonstrating detectable activity at 0.1 mg/kg of Fc-CSF-1. FIG. 3C shows the effect of 1 mg/kg dose is confirmed in a larger experimental series. The animals in this series are analysed further in subsequent studies. Organ weight studies showed that Fc-CSF-1 treatment at 1 mg/kg almost doubled the weight of the spleen and significantly increased total liver weight; whereas no effect of Fc-CSF-1 on the weights or the lung or kidney, either the whole group or segregated for male or female was observed.

The effect of Fc-CSF-1 administered to mice on blood was assessed. Results showed that Fc-CSF-1 elevates the white blood cell count and the total blood monocyte count. It was noted that there is some variation between the male and female mice, the former having higher average counts than the latter, but the effect is seen in both sexes. It was also observed that that Fc-CSF-1 increases the segmented neutrophil counts. Again, the males can be distinguished from the females. Conversely, Fc-CSF-1 had no effect on total lymphocytes

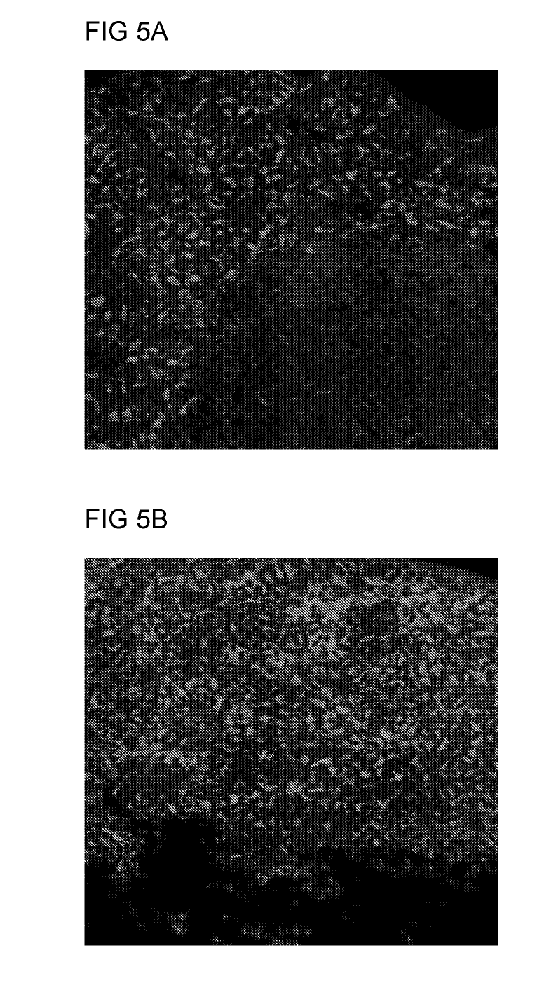

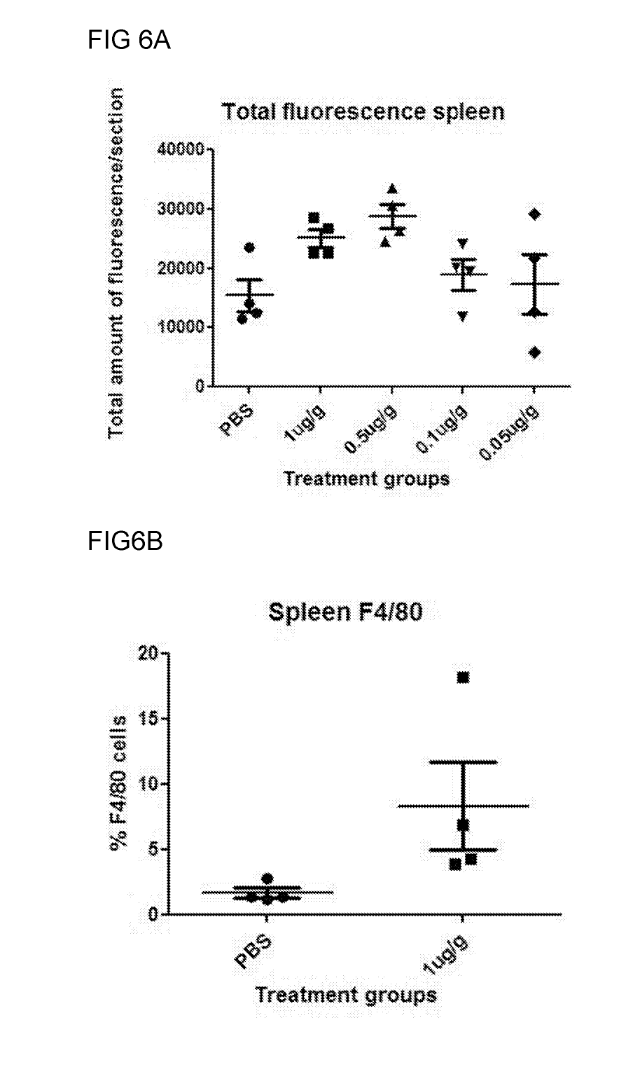

The effect of Fc-CSF-1 administered to mice on the numbers of macrophages in the spleen, detected with the csf1r-EGFP reporter, was also assessed. Note the greatly increased fluorescence in the treated FIG. 5B. This is quantitated in FIG. 6A, in a dose response curve. In FIG. 6B, the same result is demonstrated based upon immunohistochemical localisation of the macrophage-specific F4/80 antigen.

The effect of Fc-CSF-1 administered to mice on bone resorbing osteoclasts in bone was assessed. Results showed an increase in the treatment group of osteoclasts in the growth plate, zone of resorption and shaft (data not shown)

### Example 4

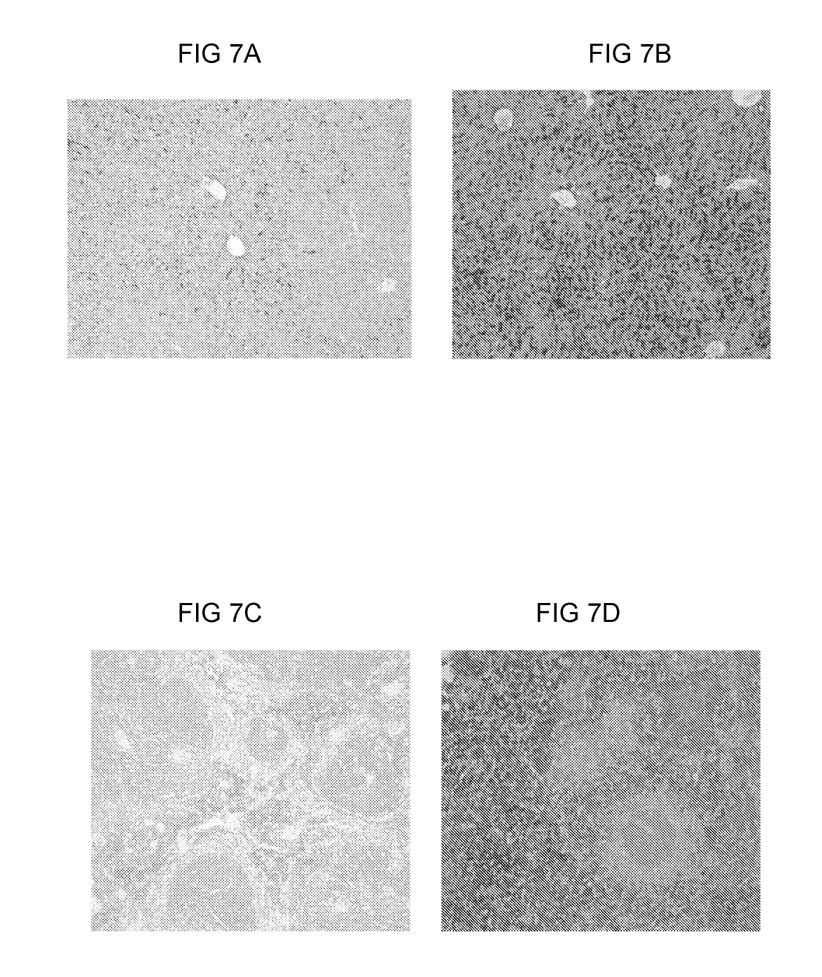

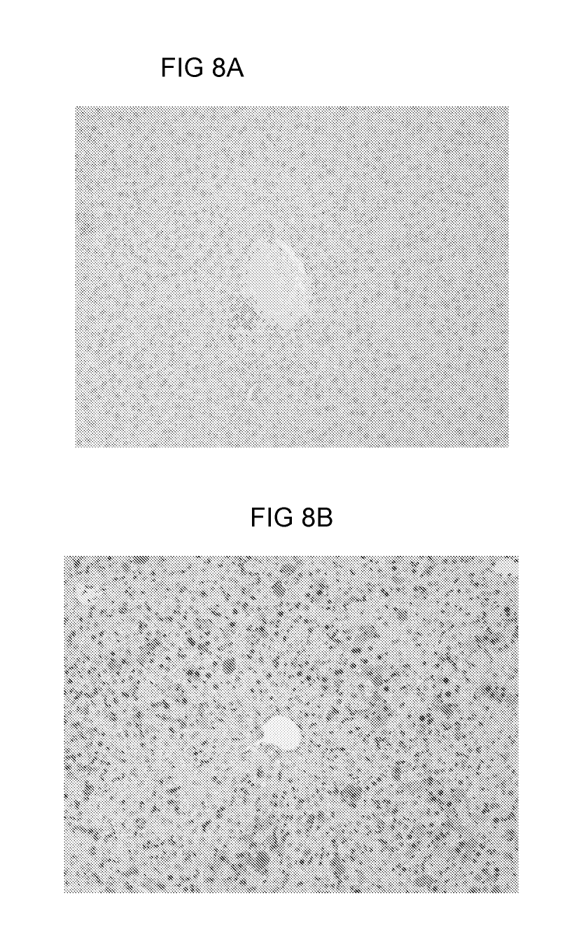

FIG. 7B shows immunostaining for the macrophage-specific F4/80 antigen in the livers of mice treated with Fc-CSF-1, demonstrating large increase in macrophage numbers over the control FIG. 7A. FIG. 7D shows immunostaining for the macrophage-specific F4/80 antigen in the spleen of mice treated with Fc-CSF-1, demonstrating large increase in macrophage numbers and also intensity of F4/80 over the control FIG. 7C. FIG. 8B shows immunostaining for proliferating cell nuclear antigen (PCNA). No staining is observed in control mouse liver FIG. 8A. FIG. 8B shows that Fc-CSF-1 causes extensive cell proliferation. Based upon size and nuclear morphology, the proliferating cells are identified as hepatocytes. In addition to the histochemical staining, the livers of control and Fc-CSF-1 treated mice have been examined using gene expression profiling on Affymetrix microarrays. The data confirm that there is a 4-8 fold increase in the abundance of known macrophage-specific genes (csf1r, emr1 (F4/80), and an even greater increase in detection of cell cycle-associated genes. Importantly, there was no evidence of induction of classical inflammatory genes such as TNF-a, IL-6 or IL-1.

### Example 5

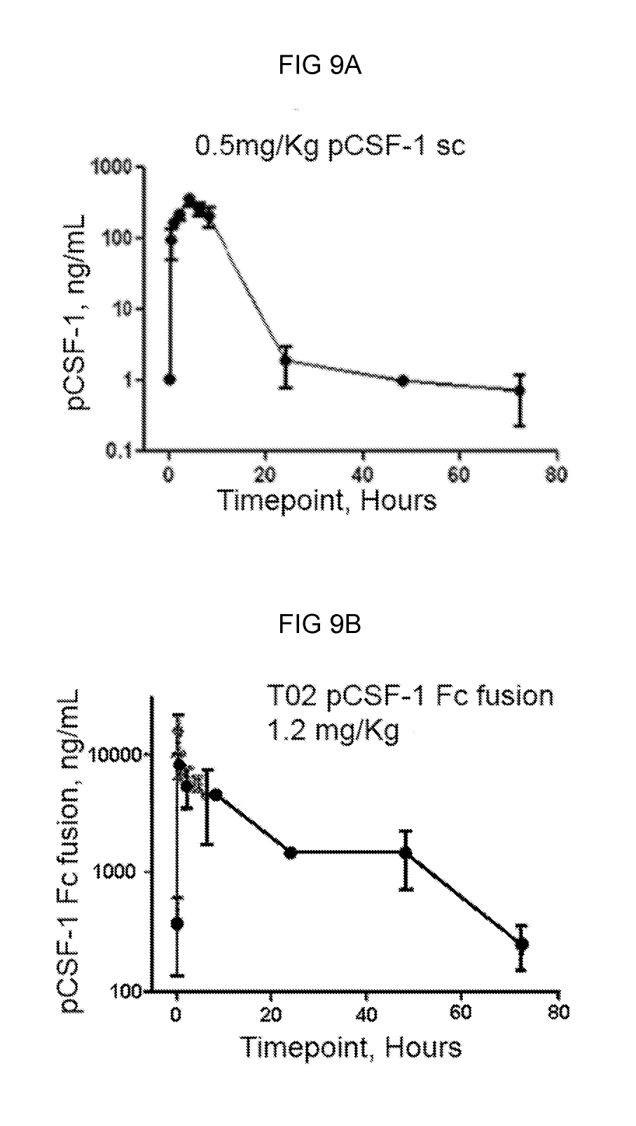

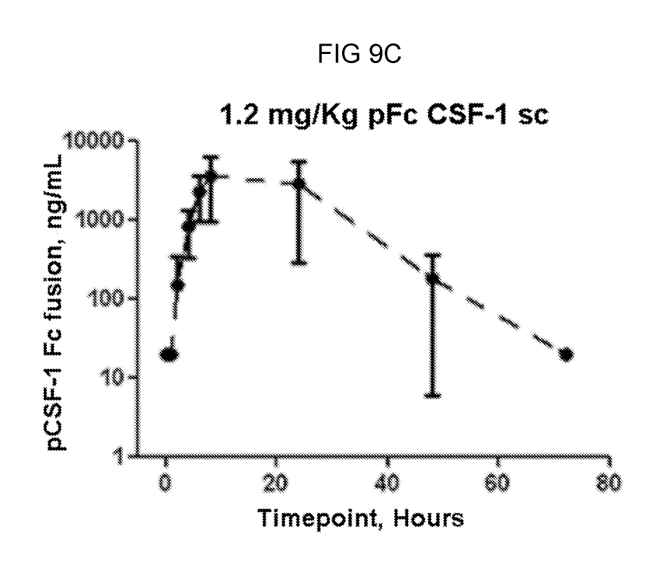

FIG. 9 demonstrates the impact of pharmacokinetics of CSF-1 administered to weaner pigs. FIG. 9A shows the clearance of unmodified CSF-1. Note that the peak plasma level obtained is only around 100 ng/ml, and it is completely cleared by 20 hours. By contrast, Fc-CSF-1 (FIGS. 9B and C) attains 100-fold higher plasma concentrations and remains elevated for up to 72 hours. A preliminary experiment on weaners determined that three treatments with 0.4 mg/kg with Fc-CSF-1 every alternative day produced a 2-3 fold increase in circulating monocyte numbers.

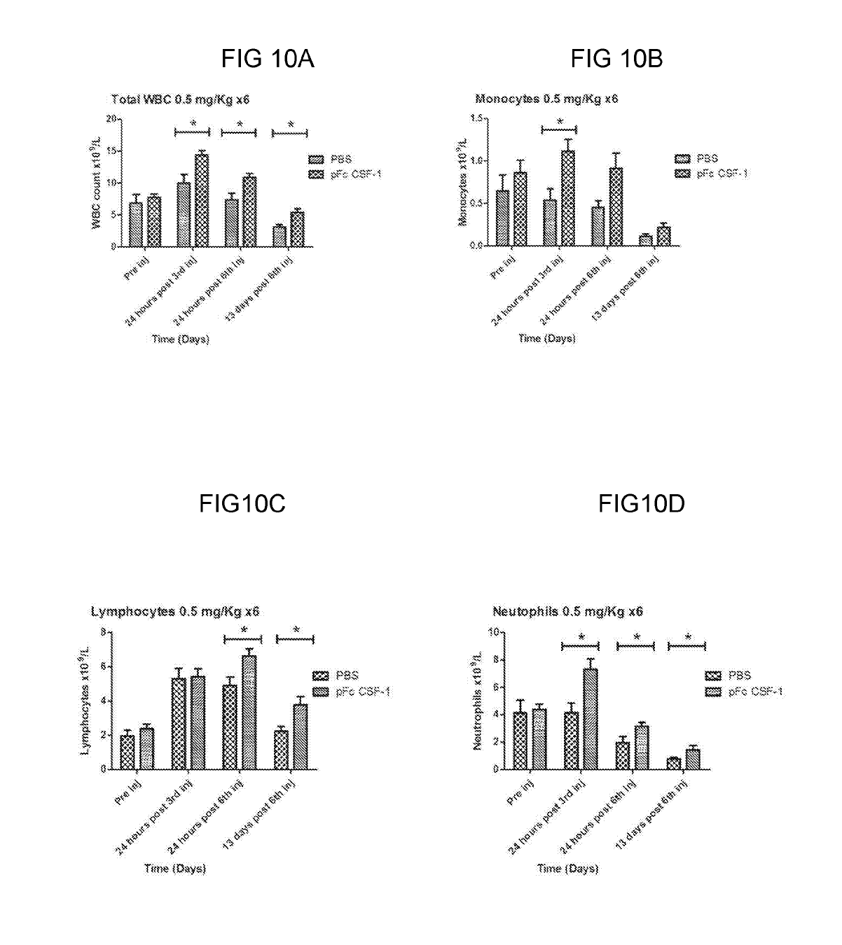

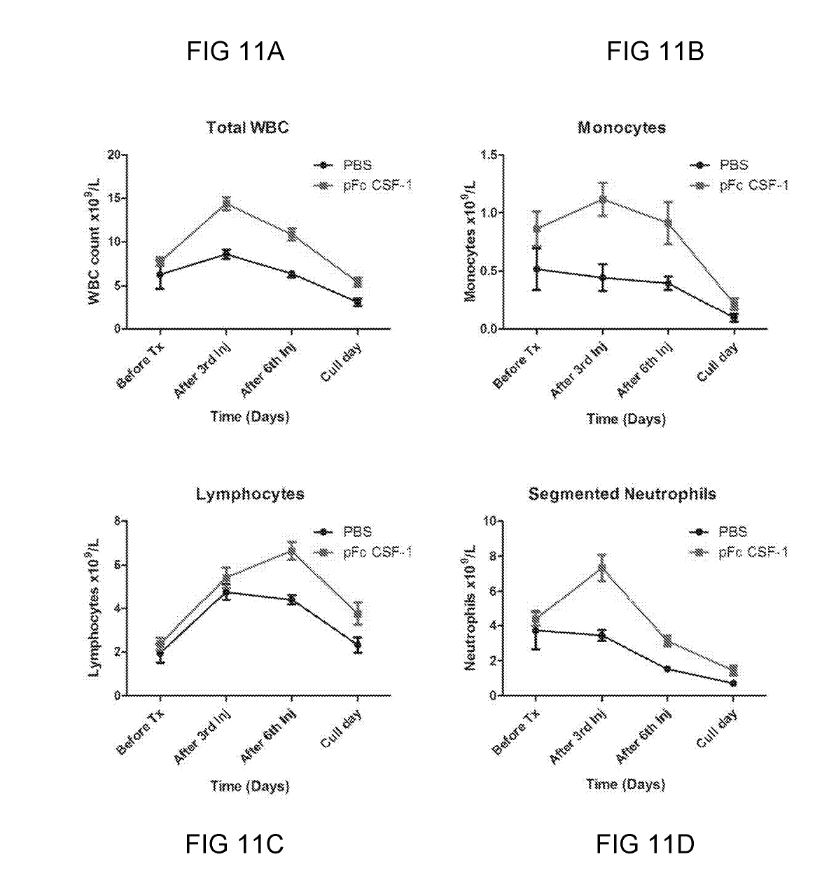

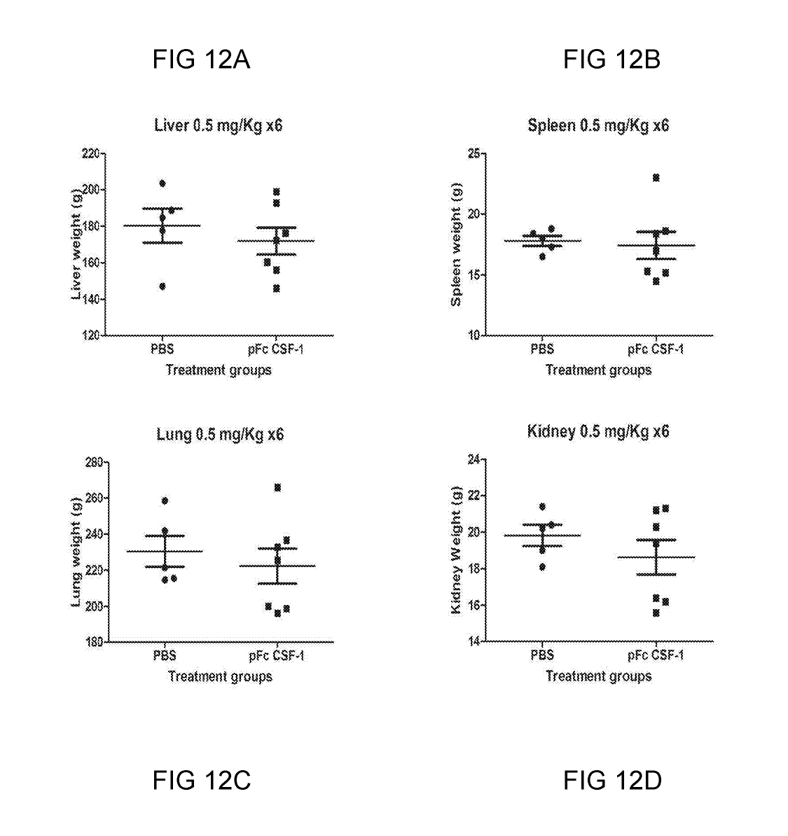

Further tests were conducted to assess Fc-CSF-1 efficacy/safety treatment trial in neonatal pigs. At the two doses tested (0.12 mg/Kg×3 and 0.5 mg/Kg×6) the Fc-CSF-1 treatment had little effect on body weight gain at any time point. A marginal decrease in weight gain was observed at the higher dose (data not shown). FIGS. 10A-D and 11A-D demonstrates the efficacy of the treatment. It was noted that in the control animals, that the total blood cell count, monocyte count and granulocyte count declines in the first two weeks of life in the pig. Fc-CSF-1 increased both the monocyte, lymphocyte and granulocyte counts significantly. FIGS. 12A-D shows that at this dose and timing, Fc-CSF-1 did not alter the organ weights measure in the liver, spleen, lung or kidney respectively, at the end of the experiment. PCNA staining revealed that there is extensive proliferation of the pig liver in the control group, which may constrain any effect at this age. Pathology report described the presence of increased numbers of histiocytes in the liver.

### Example 6

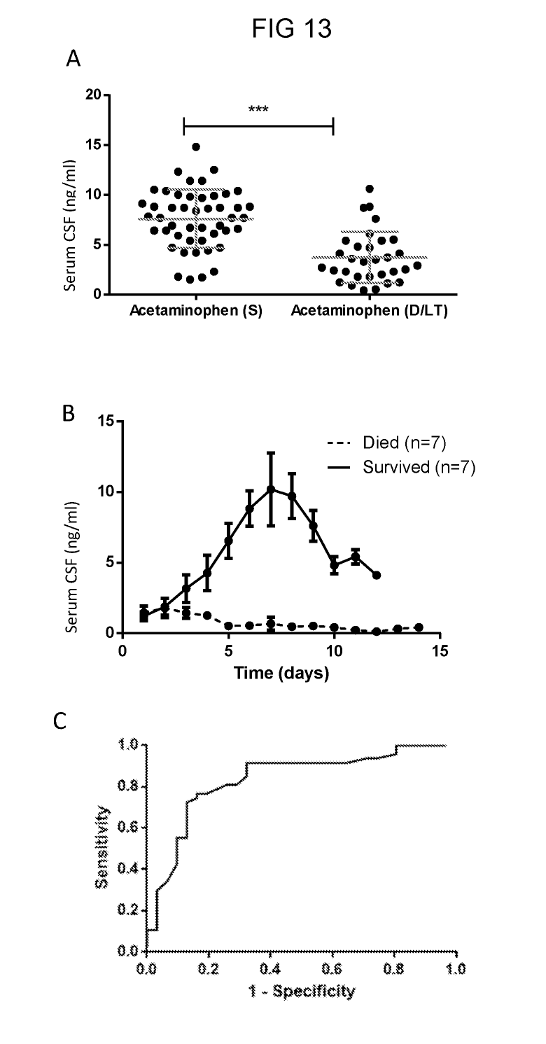

Serum macrophage colony stimulating (CSF1) was assessed using the MSD® electrochemiluminescence platform in a cohort of 78 patients presenting with acute liver failure induced by paracetamol overdose. Patients who survived showed a significantly higher serum CSF1 level than those who died or required liver transplantation (FIG. 13A). Serial samples were analysed from a subset of patients (7 survivors, 7 died/Liver transplant) demonstrating increase in serum CSF1 level in patients who survived and those who died showed a reduction in CSF1 level (FIG. 13B). CSF1 level on admission demonstrated significant predictive value for survival (ROC-AUC 0.84) (FIG. 13C).

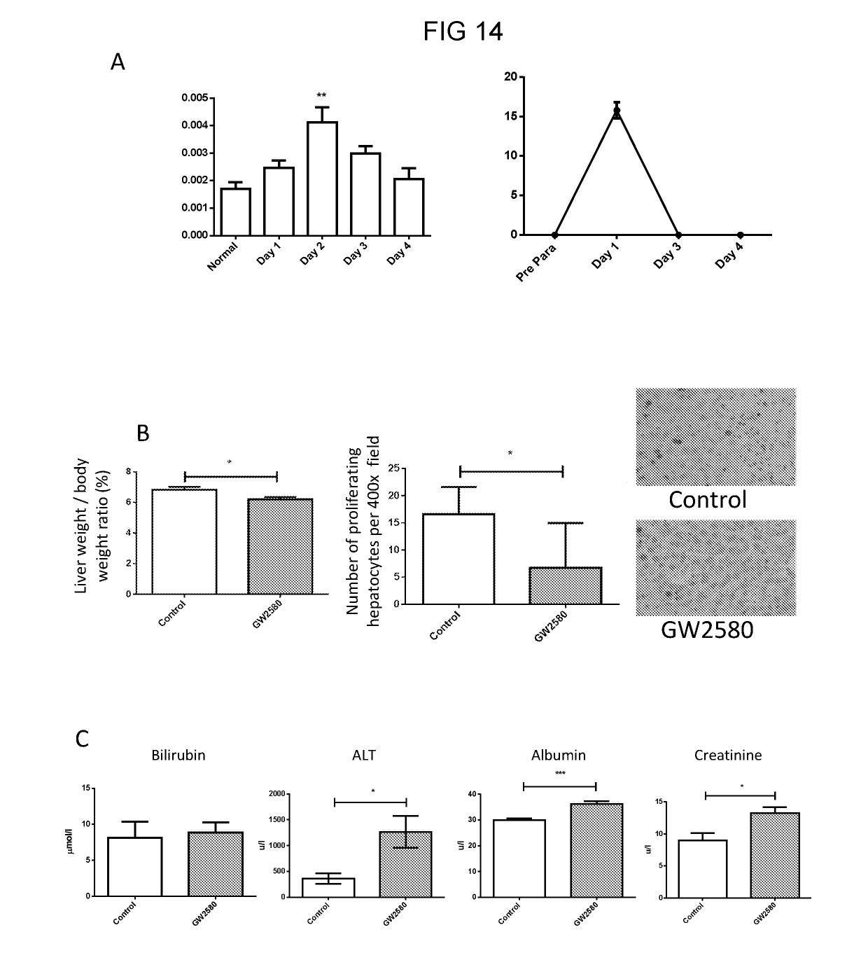

Hepatic CSF1 gene expression was assessed in mice following paracetamol intoxication (350 mg/kg paracetamol IP following overnight fast) at time points up to 4 days, showing peak CSF1 gene expression at day 2 (FIG. 14A). Serum CSF1 level assessed via Millipore Milliplex assay showed peak level at day 1 post paracetamol intoxication (FIG. 14A). Blockade of the CSF1 receptor (GW2580 180 mg/kg via gavage, LC laboratories) with paracetamol intoxication resulted in impaired liver regeneration demonstrated by reduced liver weight to body weight ratio and impaired hepatocyte proliferation at Day 3 post injury (FIG. 14B). Serum analysis is shown in FIG. 14C, demonstrating raised ALT (marker of liver injury) with CSF1 receptor blockade at Day 3 post injury.

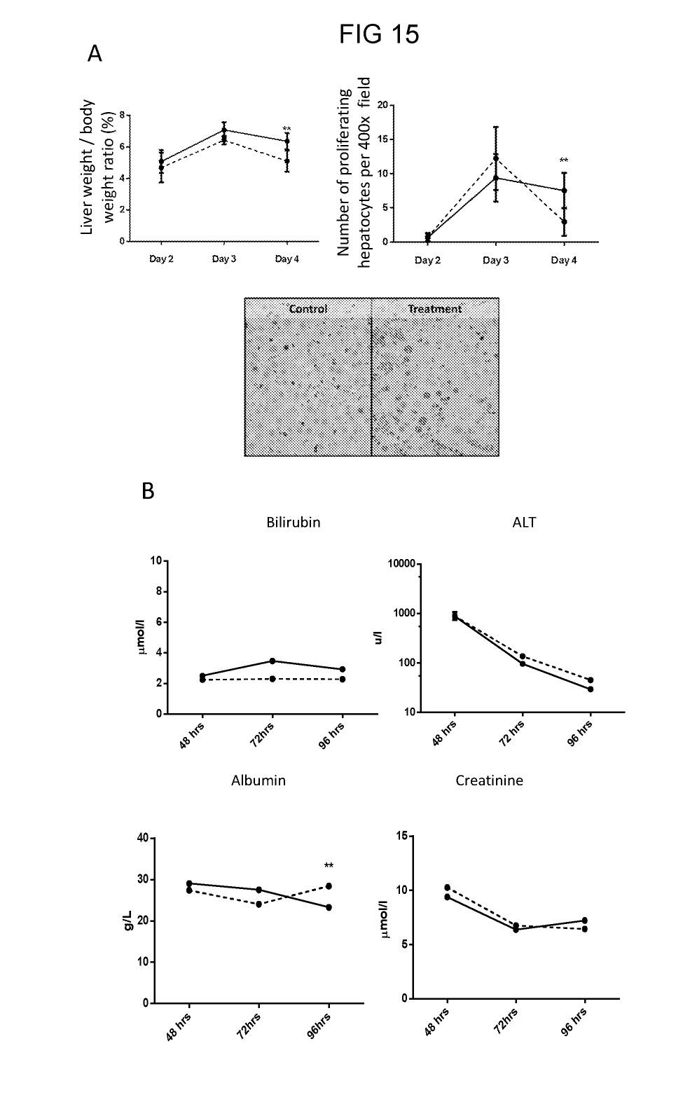

CSF1-Fc or control was administered to mice 12 hours following paracetamol intoxication significantly increasing liver weight to body weight ratio and increasing hepatocyte proliferation at Day 4 post paracetamol intoxication (FIG. 15A), serum analysis is shown in FIG. 15B.

Results demonstrate that a higher level of serum CSF1 is associated with, and predictive of, survival in humans following acute liver failure induced by paracetamol intoxication. In a mouse model of paracetamol intoxication hepatic CSF1 gene expression increases following partial hepatectomy. Blockade of the CSF1 receptor impairs liver regeneration and administration of CSF1-Fc 12 hours following paracetamol intoxication in mice can enhance regenerative parameters.

### Example 7

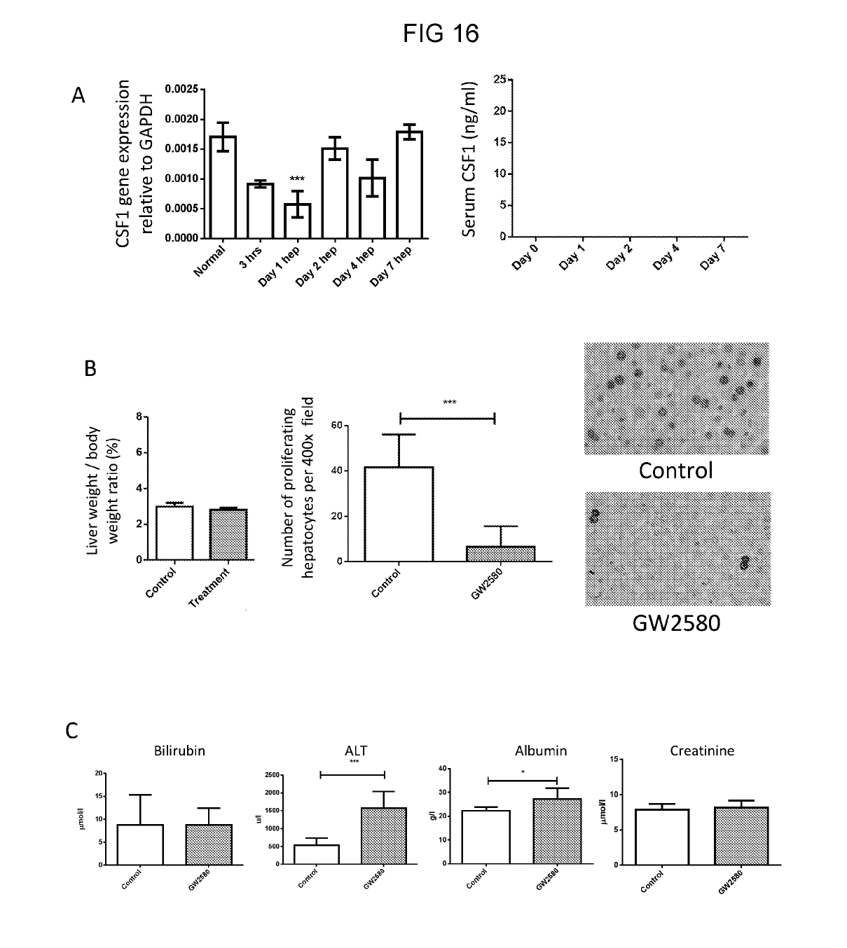

Hepatic CSF1 gene expression was assessed in mice following ⅔ partial hepatectomy at time points up to 7 days following surgery. There was an early reduction in hepatic CSF1 gene expression at Day 1 (FIG. 16A). Serum CSF1 level assessed via Millipore Milliplex assay was undetectable in this mouse model (FIG. 16A). However blockade of the CSF1 receptor (GW2580 180 mg/kg via gavage, LC laboratories) with ⅔ partial hepatectomy resulted in impaired liver regeneration demonstrated by markedly impaired hepatocyte proliferation at Day 3 (FIG. 16B). Serum analysis is shown in FIG. 16C, demonstrating raised ALT (marker of liver injury) with CSF1 receptor blockade.

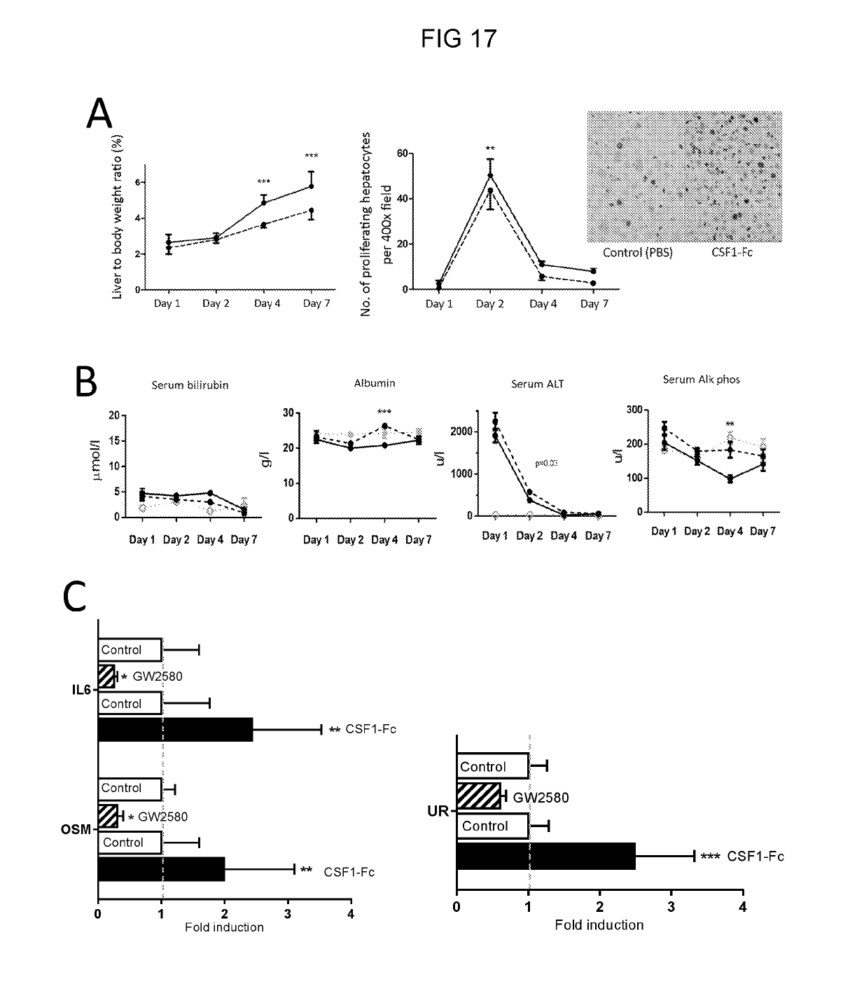

CSF1-Fc or control was administered to mice immediately following ⅔ partial hepatectomy significantly increasing liver weight to body weight ratio and increasing hepatocyte proliferation (FIG. 17A). Serum analysis is shown in FIG. 17B. CSF1-Fc administration significantly enhanced gene expression of pro-regenerative cytokines 116 and oncostatin M (OSM) whereas CSF1 receptor inhibition with GW2580 resulted in a significant reduction in their expression at day 2 following partial hepatectomy. Urokinase receptor (UR), which is involved in growth factor activation was significantly elevated with CSF1-Fc administration with a reduction in urokinase receptor expression with CSF1 receptor blockade (GW2580) (FIG. 17C).

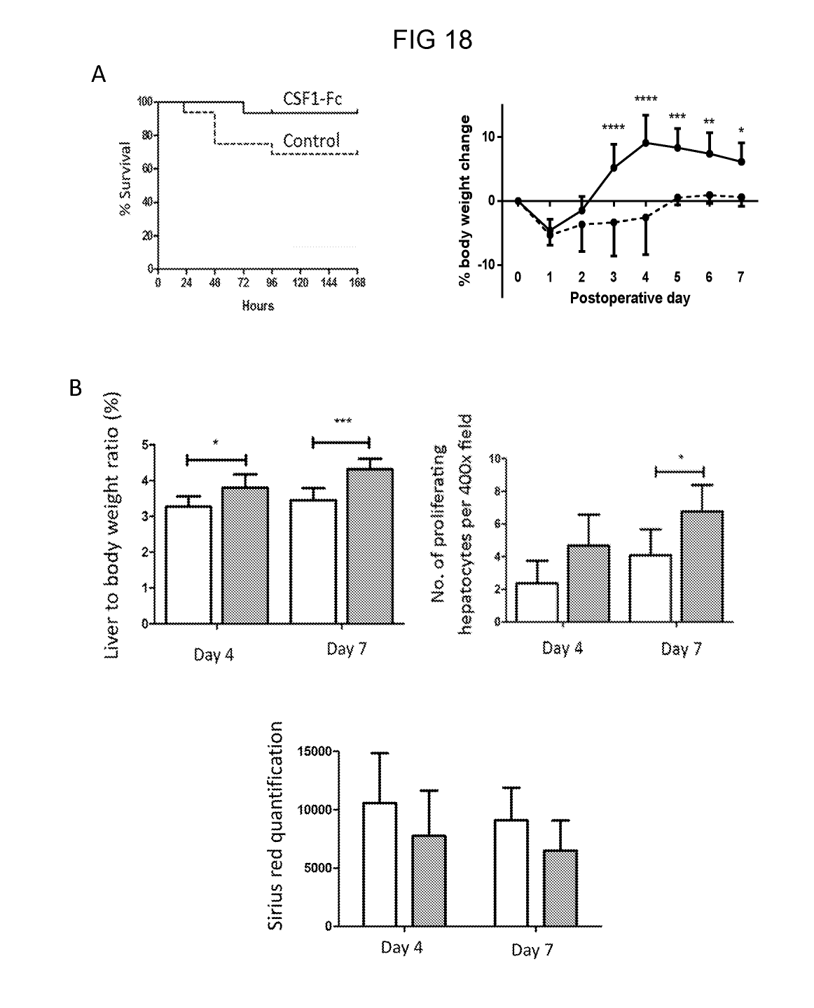

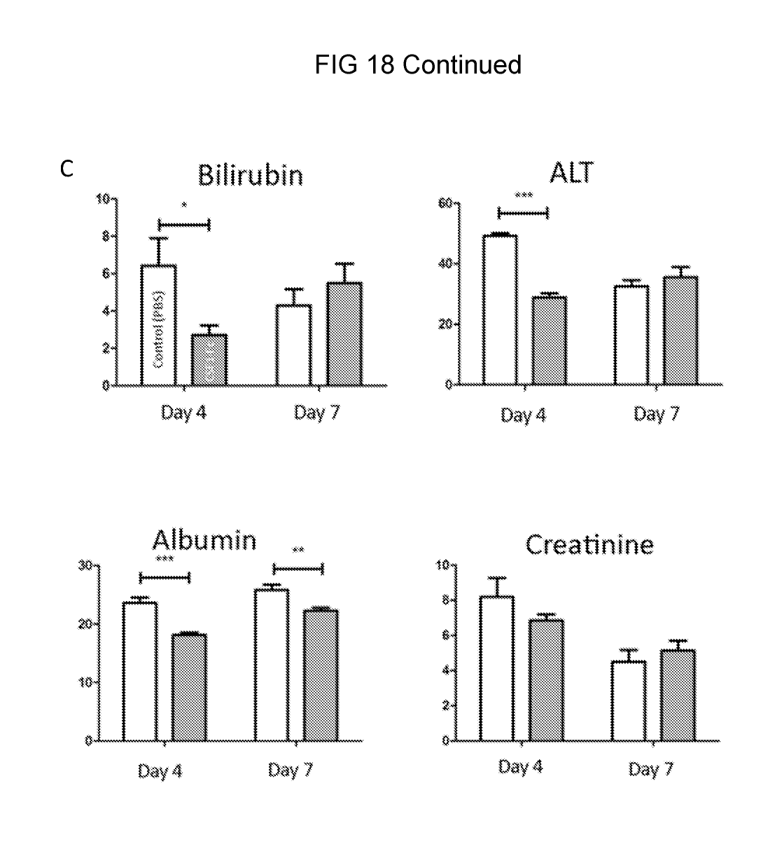

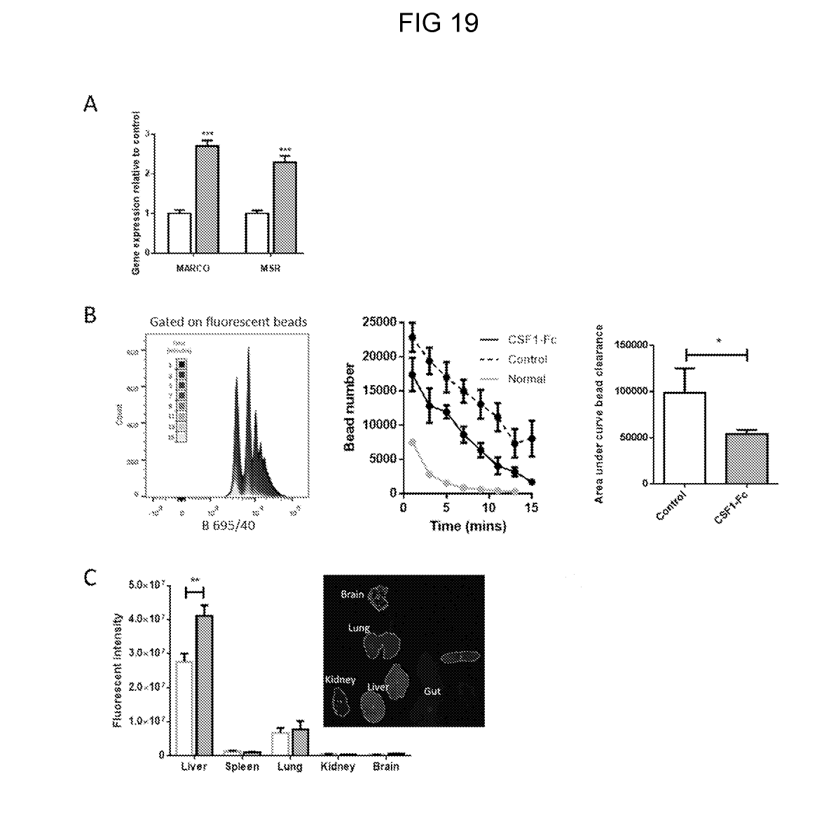

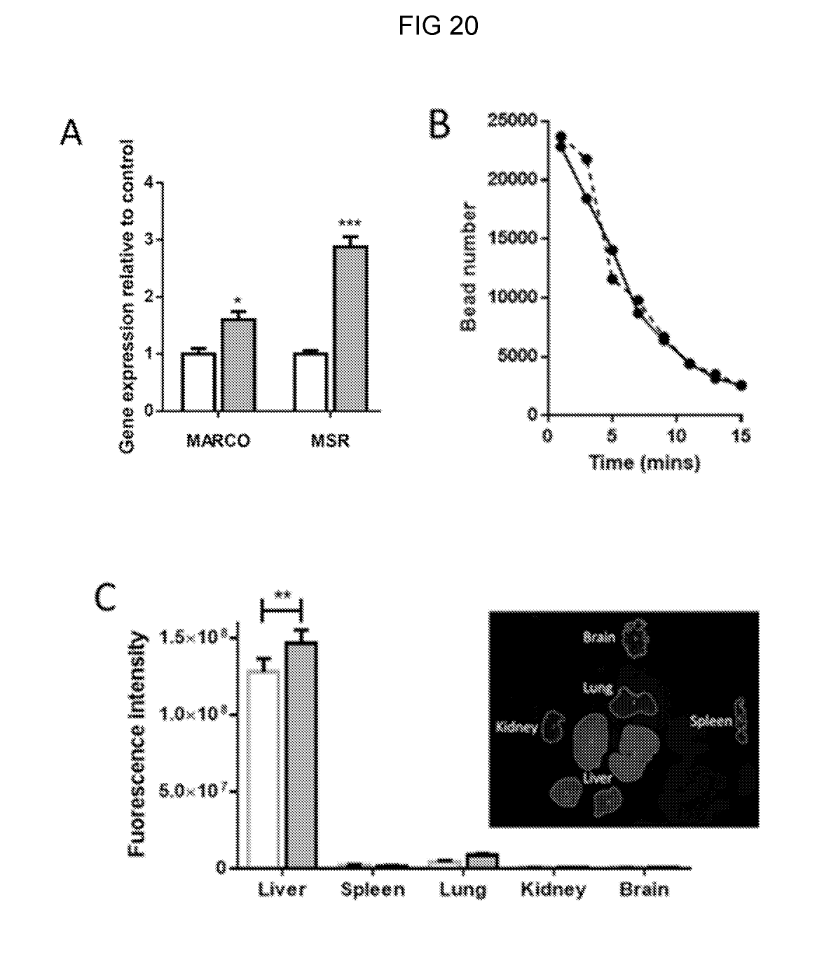

CSF1-Fc or control was administered to mice immediately following ⅔ partial hepatectomy on a background of 8 weeks carbon tetrachloride induced chronic liver injury (1 mcl/g carbon tetrachloride/mouse 2×/week). There was a trend to improved survival with CSF1-Fc treatment and significant increase in body weight (FIG. 18A). Liver weight to body weight ratio and number of proliferating hepatocytes was increased significantly and there was a trend to reduction in fibrosis assessed by Sirius red quantification (FIG. 185B). Serum parameters are shown in FIG. 18C demonstrating significant reduction in bilirubin and ALT at day 4 post hepatectomy with CSF1-Fc treatment.

In contrast to the situation in paracetamol intoxication it was found that CSF1 gene expression and serum level did not rise following partial hepatectomy. However blockade of the CSF1 receptor significantly impaired liver regeneration. Administration of CSF1-Fc significantly enhanced markers of regeneration in models of partial hepatectomy in the normal and chronically injured mouse liver.

### Example 8

### CSF1-Fc Enhances Hepatic Phagocytic Ability Following Injury

### Background

Situated downstream of the gut, the liver is constantly exposed to pathogenic material and it is in this context it performs detoxification and innate immune functions central to maintaining homeostasis. Hepatic macrophages represent the largest population of macrophages in direct circulatory contact, playing a major role in phagocytosis of pathogenic and other insoluble material. Liver injury places substantial regenerative demand on the liver, dramatically reducing phagocytic capacity and immune function[1, 2]. At present there are no available therapies to enhance hepatic phagocytic ability.

### Methods

C57Bl6 male mice (8-10 weeks) underwent either partial hepatectomy (⅔ resection) or paracetamol intoxication (350 mg/kg intraperitoneal following overnight fast). CSF1-Fc was administered as previous (0.75 mg/kg). Gene analysis was performed using Qiagen Quantitect Primers (MSR1 and MARCO) and related to GAPDH level for each sample. For the phagocytosis assay mice were anaesthetised with 2% isolfluorane and the inferior vena cava was cannulated. 0.1 mls of 5000 IU/ml heparin solution was infused to prevent blockage of the catheter. 100 μl of red fluorescent bead solution (1:5 Latex beads 1.0 μm, fluorescent red, SIGMA-ALDRICH®) was infused through the cannula (1:2 solution for assay following paracetamol injury). 20 mcl of blood was removed from the cannula every two minutes starting from 1 minute post injection for 15 minutes. Blood was immediately fixed with 300 μl FACS-Lysing solution (BD Biosceinces). After 15 minutes mice were perfused with 15 mls 0.9% saline through the IVC cannula after dividing the portal vein for outflow. Organs were then removed (Liver, spleen, lungs, kidney, brain) and imaged with a Kodak In-Vivo Multispectral FX image station (Excitation: 550 nm; Emission: 600 nm; Exposure 1 sec; f-stop 2.8). Subsequently blood samples were analysed using a LSR-Fortessa™flow cytometer (BD Biosciences) with fluorescent beads detected on the blue channel (B695/40) by a 1 minute sample collection on low flow rate setting.

### Findings

### CSF1-Fc and Partial Hepatectomy

Gene analysis of whole liver revealed upregulation of genes associated with phagocytosis, MSR 1 (macrophage scavenger receptor 1) and MARCO (macrophage receptor with collagenous structure) at day 2 following partial hepatectomy with CSF1-Fc treatment. CSF1 receptor blockade resulted in a reciprocal decrease in expression of these genes.

Treatment with CSF1-Fc following partial hepatectomy dramatically enhanced the phagocytic ability of the liver as assessed by clearance from the circulation of fluorescent latex microbeads (Fig B). Fluorescent imaging of whole liver revealed increased fluorescent intensity of the liver consistent with the presence of enhanced phagocytosis (Figure C). Fluorescent imaging of other organs revealed that the liver was the dominant site of bead uptake.

### CSF1-Fc and Paracetamol Intoxication

Gene analysis of whole liver revealed upregulation of genes associated with phagocytosis, MSR 1 (macrophage scavenger receptor 1) and MARCO (macrophage receptor with collagenous structure) at day 2 following paracetamol intoxication.

The loss of hepatic tissue was markedly less severe in the paracetamol intoxication model compared to partial hepatectomy and consistent with this we did not notice enhanced clearance of the latex microbeads from the circulation in the paracetamol model. We did however see a significant increase in hepatic fluorescence following ex vivo imaging, suggestive of increased phagocytic capacity.

### CONCLUSION

These findings demonstrate that following liver injury the phagocytic ability of the liver can be enhanced by treatment with CSF1-Fc.

### Example 9

Partial hepatectomy and the effects of serum CSF1 level in humans was assessed as follows.

### Methods

Serum macrophage colony stimulating (CSF1) was assessed using the MSD® electrochemiluminescence platform in a cohort of 55 patients who underwent partial hepatectomy. Serum samples were taken preoperatively and on Day 1 and Day 3 postoperatively.

### Findings

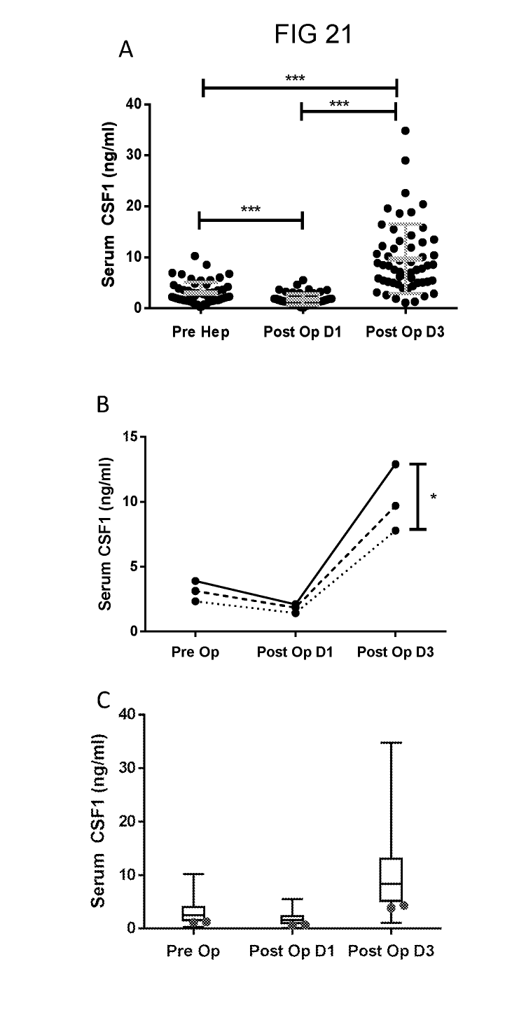

In humans following partial hepatectomy a significant decrease in serum CSF1 level was seen at Day1 with a subsequent increase in CSF1 level on day 3 (FIG. 21 A). The greatest increase was seen in patients who had the greatest number of segments removed (more than 5 segments compared to less than 3) (FIG. 21 B). Of the whole cohort 2 patients developed postoperative liver failure. The CSF1 level of these patients was in the lowest quartile (FIG. 21 C).

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### REFERENCES

1. Canalese, J., et al., Reticuloendothelial system and hepatocytic function in fulminant hepatic failure. Gut, 1982. 23(4): p. 265-9.
2. Schindl, M. J., et al., The adaptive response of the reticuloendothelial system to major liver resection in humans. Ann Surg, 2006. 243(4): p. 507-14.

HUMANCSF-1 SEQUENCE

Full length (residues 1-554):
(SEQ ID NO: 3)
MTAPGAAGRCPPTTWLGSLLLLVCLLASRSITEEVSEYCSHMIGSGHL
QSLQRLIDSQMETSCQITFEFVDQEQLKDPVCYLKKAFLLVQDIMEDT
MRFRDNIPNAIAIVQLQELSLRLKSCFTKDYEEHDKACVRTFYETPLQ
LLEKVKNVFNETKNLLDKDWNIFSKNCNNSFAECSSQDVVTKPDCNCL
YPKAIPSSDPASVSPHQPLAPSMAPVAGLTWEDSEGTEGSSLLPGEQP
LHTVDPGSAKQRPPRSTCQSFEPPETPVVKDSTIGGSPQPRPSVGAFN
PGMEDILDSAMGTNWVPEEASGEASEIPVPQGTELSPSRPGGGSMQTE
PARPSNFLSASSPLPASAKGQQPADVTGTALPRVGPVRPTGQDWNHTP
QKTDHPSALLRDPPEPGSPRISSLRPQGLSNPSTLSAQPQLSRSHSSG
SVLPLGELEGRRSTRDRRSPAEPEGGPASEGAARPLPRFNSVPLTDTG
HERQSEGSFSPQLQESVFHLLVPSVILVLLAVGGLLFYRWRRRSHQEP
QRADSPLEQPEGSPLTQDDRQVELPV
Sequence with signal peptide removed
(residues 33-554):
(SEQ ID NO: 4)
EEVSEYCSHMIGSGHLQSLQRLIDSQMETSCQITFEFVDQEQLKDPVC
YLKKAFLLVQDIMEDTMRFRDNTPNAIAIVQLQELSLRLKSCFTKDYE
EHDKACVRTFYETPLQLLEKVKNVFNETKNLLDKDWNIFSKNCNNSFA
ECSSQDVVTKPDCNCLYPKAIPSSDPASVSPHQPLAPSMAPVAGLTWE
DSEGTEGSSLLPGEQPLHTVDPGSAKQRPPRSTCQSFEPPETPVVKDS
TIGGSPQPRPSVGAFNPGMEDILDSAMGTNWVPEEASGEASEIPVPQG
TELSPSRPGGGSMQTEPARPSNFLSASSPLPASAKGQQPADVTGTALP
RVGPVRPTGQDWNHTPQKTDHPSALLRDPPEPGSPRISSLRPQGLSNP
STLSAQPQLSRSHSSGSVLPLGELEGRRSTRDRRSPAEPEGGPASEGA
ARPLPRFNSVPLTDIGHERQSEGSFSPQLQESVFHLLVPSVILVLLAV
GGLLFY RWRRRSHQEPQRADSPLEQPEGSPLTQDDRQVELPV
Active Fragment, i.e. residues 33-182:
(SEQ ID NO: 5)
EEVSEYCSHMIGSGHLQSLQRLIDSQMETSCQITFEFVDQEQLKDPVC
YLKKAFLLVQDIMEDTMRFRDNTPNAIAIVQLQELSLRLKSCFTKDYE
EHDKACVRTFYETPLQLLEKVKNVFNETKNLLDKDWNIFSKNCNNSFA
ECSSQD

1. A biologically active fragment of CSF-1 protein or a homolog or a variant or derivative thereof for use in enhancing liver regeneration and/or restoring liver function and/or modulating liver homeostasis.

2. A nucleic acid encoding the biologically active fragment of CSF-1 protein or a homolog or a variant or derivative thereof of claim 1.

3. A fusion protein comprising:
(i) a biologically active fragment of CSF-1 or a homolog or a variant or a derivative thereof; and
(ii) a biologically active antibody fragment.

4. The fusion protein according to claim 3 wherein, the biologically active fragment of CSF-1 protein is residues 33-182 of human CSF-1 or a biologically active portion thereof, or a biological equivalent fragment of CSF-1 from any mammalian species.

5. The fusion protein of claim 3 wherein the biologically active fragment of CSF-1 protein or a homolog or a variant or derivative thereof is either native/naturally occurring or is recombinant.

6. The fusion protein according to claim 3 wherein, the antibody is an immunoglobulin selected from the group comprising IgA, IgD, IgE, IgG and IgM.

7. (canceled)

8. The fusion protein according to claim 3 wherein, the antibody fragment is selected from the group comprising F(ab′)2, Fab′, Fab, Fv, Fc and rIgG.

9. (canceled)

10. The fusion protein according to claim 3 wherein, the biologically active fragment of CSF-1 or a homolog or a variant or derivative thereof and the biologically active antibody fragment of the fusion protein are covalently linked directly or through a linker moiety.

11. An isolated nucleic acid encoding the fusion protein according to claim 3.

12. A vector comprising the isolated nucleic acid of claim 11.

13. A host cell comprising the vector of claim 12.

14. A method of making the fusion protein according to claim 3, the method comprising:
(i) culturing the host cell of claim 13; and
(ii) collecting the fusion protein from said culture.

15. A composition comprising:
(a) at least one fusion protein comprising (i) a biologically active fragment of CSF-1 or a homolog or a variant or a derivative thereof; and (ii) a biologically active antibody fragment; and
(b) a pharmaceutically acceptable carrier, excipient or diluent.

16. (canceled)

17. A composition comprising the nucleic acid of claim 11, a pharmaceutically acceptable carrier, excipient or diluents.

18. Use of a fusion protein comprising:
(i) a biologically active fragment of CSF-1 or a homolog or a variant or a derivative thereof; and
(ii) a biologically active antibody fragment
for enhancing liver regeneration and/or restoring liver function and/or modulating liver homeostasis.

19. Use of the fusion protein according to claim 3 for the manufacture of a medicament for enhancing liver regeneration and/or restoring liver function and/or modulating liver homeostasis.

20. A method of treatment for an individual suffering from liver cancer and who is to undergo surgery, the method comprising administering the fusion protein according to claim 3 before, during or after the surgical procedure.

21. A method of treatment for an individual who is to undergo liver transplant surgery, the method comprising administering the fusion protein according to claim 3 before, during or after the surgical procedure.

22. A kit comprising one or more containers having pharmaceutical dosage units comprising an effective amount of the fusion protein according to claim 3, wherein the container is packaged with optional instructions for the use thereof.

23. Use of the fusion protein according to the nucleic acid of claim 11 for the manufacture of a medicament for enhancing liver regeneration and/or restoring liver function and/or modulating liver homeostasis.

24. A method of treatment for an individual suffering from liver cancer and who is to undergo surgery, the method comprising administering the nucleic acid of claim 11 before, during or after the surgical procedure.

25. A method of treatment for an individual who is to undergo liver transplant surgery, the method comprising administering the fusion protein according to the nucleic acid of claim 11 before, during or after the surgical procedure.

26. A kit comprising one or more containers having pharmaceutical dosage units comprising an effective amount of the nucleic acid of claim 11, wherein the container is packaged with optional instructions for the use thereof.


SEQUENCE LISTING (5 sequences)

SEQ ID NO: 1 - protein, 154 aa, Sus scrofa
SENCSHMIGDGHLKVLQQLIDSQMETSCQIAFEFVDQEQLTDPVCYLKKAFLQVQDILDETMRFRDNTPNANVIVQLQELSLRLNSCFTKDYEEQDKACVRTFYETPLQLLEKIKNVFNETKNLLKKDWNIFSKNCNNSFAKCSSQHERQPEGR

SEQ ID NO: 2 - protein, 230 aa, Sus scrofa
GTKTKPPCPICPGCEVAGPSVFIFPPKPKDTLMISQTPEVTCVVVDVSKEHAEVQFSWYVDGVEVHTAETRPKEEQFNSTYRVVSVLPIQHQDWLKGKEFKCKVNNVDLPAPITRTISKAIGQSREPQVYTLPPPAEELSRSKVTVTCLVIGFYPPDIHVEWKSNGQPEPEGNYRTTPPQQDVDGTFFLYSKLAVDKARWDHGETFECAVMHEALHNHYTQKSISKTQGK

SEQ ID NO: 3 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 4 - protein, 522 aa, Homo sapiens
EEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 5 - protein, 150 aa, Homo sapiens
EEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQD